(19) United States
(12) Patent Application Publication
(10) Pub. No.: US 2016/0264918 A1
(43) Pub. Date: Sep. 15, 2016

(54) Cell Culture Apparatus

(71) Applicant: HITACHI HIGH-TECHNPLOGIES CORPORATION, Tokyo (JP)

(72) Inventors: Akihiro SHIMASE, Tokyo (JP); Kazumichi IMAI, Tokyo (JP); Eiichiro TAKADA, Tokyo (JP); Sadamitsu ASO, Tokyo (JP)

(21) Appl. No.: 15/030,108

(22) PCT Filed: Sep. 2, 2014
(86) PCT No.: PCT/JP2014/073073

§ 371 Date: Apr. 18, 2016

(30) Foreign Application Priority Data
Nov. 7, 2013 (JP) 2013-231257

(51) Int. Cl. C12M 1/00

(52) U.S. Cl. CPC C12M 23/58; C12M 23/40; C12M 29/00

(57) ABSTRACT

A cell culture apparatus in which liquid can be supplied uniformly/homogeneously among a plurality of culture containers while keeping cost low. In this cell culture apparatus, a plurality of sealed culture containers (1) having fluid introduction ports and discharge ports are provided, and the plurality of culture containers (1) are connected in parallel, forming a single closed culture system. The cell culture apparatus has a single flow channel switching mechanism (8) for switching a plurality of flow channels connected to the plurality of culture containers (1). The flow channel switching mechanism (8) makes the flow channel resistance of one individual flow channel from among the individual flow channels branching and connecting to the culture containers (1) less than the flow channel resistance of the remainder of the individual flow channels. The flow channel switching mechanism (8) forms an individual flow channel having a small flow channel resistance by placing a single flow channel switching member in the closed culture system and changing the orientation and/or position of the flow channel switching member.

### TECHNICAL FIELD

The present invention relates to a cell culture apparatus to culture a cell using a culture container.

### BACKGROUND ART

Conventionally, work of culturing a cell has been performed by manual labor of a skilled worker under a strict manufacturing process in a clean room which is unlimitedly sterilized. Thus, enormous labor cost and facility cost are required in the case of culturing the cell in large amount, which causes a great barrier to industrialization.

It is possible to automate a series of culture operations, which has been performed by the manual labor, using a robot, and to reduce the labor cost. However, it is not necessarily avoidable to a risk of contamination from outside since there is an operation to open an inside of a culture container, for example, in order to replace a culture medium by opening a lid of the culture container. Thus, there is a need of installing the entire system including the robot in a large-scale clean room, and it is difficult to significantly reduce the facility cost.

Accordingly, a system has been proposed which cultures a cell by forming a single sealed system by connecting a liquid bag with a culture medium therein and a culture container or the like (hereinafter, referred to as a closed culture system), and performing replacement of the culture medium or the like inside the closed culture system. An example thereof is described in JP2011-142837 A (PTL 1), for example. Accordingly, the cleanliness of a location in which the system has been installed may be kept while enabling the exclusion of the risk of contamination from the outside, and the significant reduction of the facility cost becomes possible.

In addition, a method of controlling liquid supply in a closed culture system which has a plurality of culture containers is described in JP 2009-125027 A (PTL 2), for example.

### CITATION LIST

### Patent Literature

PTL 1: JP 2011-142837 A

PTL 2: JP 2009-125027 A

### SUMMARY OF INVENTION

### Technical Problem

In general, it is considered to increase a culture area in order to increase a yield of cell culture. At the time, dozens or hundreds of types of culture containers having different sizes may be prepared to selectively use the culture container according to the yield, which is diseconomy. Therefore, it is economical to prepare culture containers having a limited number of types of sizes, and to change the yield by changing the number of the containers. Such a technique of simultaneously culturing the same cells in a plurality of the culture containers is often performed in order to increase the yield. The important point at this time is that there is no variation in quality of results of the culture. In order for this, it is important to uniformly perform a culture operation such as culture medium replacement among the plurality of culture containers. This is also applied to a closed culture system which has a plurality of culture containers.

A method of connecting culture containers in series and a method of connecting culture containers in parallel are considered in order to form a single closed culture system with the plurality of culture containers.

An example of advantages of the method of connecting the culture containers in series is that it is easy to control the liquid supply such as the culture medium replacement. Since there is no branch, the liquid reliably and uniformly spreads to each culture container although being on a rotating basis. However, there is a problem that the quality of liquid is easily degraded as going down from the upstream to the downstream in the case of the series connection. It is also necessary to obtain uniformity in terms of quality as well as of amount of the supplied liquid in order to minimize a variation in the culture quality. In addition, there is another problem that, in a case in which contamination occurs at the upstream, the downstream is also contaminated.

It is possible to solve the above-described problem when the culture containers are connected in parallel to separately supply the liquid. A method of supplying a liquid by switching culture containers (set), which are connected in parallel, using a valve is described in PTL 2, described above. Accordingly, a homogeneous liquid supply with respect to the plurality of culture containers becomes possible, but there is a problem that the number of the valves increases in the case of increasing the number of branches when the valve is provided for each branch, which causes an increase of cost.

Therefore, the present invention has been made in view of the above-described problems, and a representative object thereof is to provide a cell culture apparatus which enables a uniform and homogeneous liquid supply among a plurality of culture containers while keeping cost low.

Other objects and novel characteristics in addition to the above-described ones of the present invention will be apparent from description of the present specification and the attached drawings.

### Solution to Problem

An overview of representatives of the invention to be disclosed in the present application will be simply described as follows.

That is, a representative cell culture apparatus is a cell culture apparatus in which a plurality of sealed culture containers having fluid introduction ports and discharge ports are provided, and the plurality of culture containers are connected in parallel, forming a single closed culture system. The cell culture apparatus has a single flow channel switching mechanism for switching a plurality of flow channels connected to the plurality of culture containers.

More preferably, the flow channel switching mechanism in the cell culture apparatus makes a flow channel resistance of one individual flow channel from among the individual flow channels branching and being connected to the culture containers less than the flow channel resistance of the remainder of the individual flow channels.

More preferably, the flow channel switching mechanism in the cell culture apparatus forms an individual flow channel having a small flow channel resistance by placing a single flow channel switching member in the closed culture system and changing at least one of an orientation and a position of the flow channel switching member.

### Advantageous Effects of Invention

An effect that can be obtained by the representatives of the invention to be disclosed in the present application will be simply described as follows.

That is, the representative effect is that it is possible to provide a cell culture apparatus which enables a uniform and homogeneous liquid supply among a plurality of culture containers while suppressing cost.

### BRIEF DESCRIPTION OF DRAWINGS

FIG. 1 is a diagram illustrating an example of an overview of a cell culture apparatus according to Embodiment 1 of the present invention.

FIGS. 2(a) and 2(b) are diagrams illustrating examples of a structure of a flow channel switching mechanism according to Embodiment 1 of the present invention.

FIGS. 3(a) and 3(b) are diagrams illustrating examples of a flow channel resistance of the flow channel switching mechanism according to Embodiment 1 of the present invention.

FIGS. 4(a) and 4(b) are diagrams illustrating modified examples of the structure of the flow channel switching mechanism according to Embodiment 1 of the present invention.

FIGS. 5(a) to 5(d) are diagrams illustrating modified examples of the structure of the flow channel switching mechanism according to Embodiment 1 of the present invention.

FIGS. 6(a) and 6(b) are diagrams illustrating modified examples of the structure of the flow channel switching mechanism according to Embodiment 1 of the present invention.

FIGS. 7(a) to 7(d) are diagrams illustrating modified examples of the structure of the flow channel switching mechanism according to Embodiment 1 of the present invention.

FIGS. 8(a) to 8(c) are diagrams illustrating modified examples of the structure of the flow channel switching mechanism according to Embodiment 1 of the present invention.

FIGS. 9(a) and 9(b) are diagrams illustrating modified examples of the structure of the flow channel switching mechanism according to Embodiment 1 of the present invention.

FIGS. 10(a) and 10(b) are diagrams illustrating modified examples of the structure of the flow channel switching mechanism according to Embodiment 1 of the present invention.

FIG. 11 is a diagram illustrating a modified example of the structure of the flow channel switching mechanism according to Embodiment 1 of the present invention.

FIGS. 12(a) and 12(b) are diagrams illustrating examples of a method of switching a flow channel switching member according to Embodiment 1 of the present invention.

FIGS. 13(a) and 13(b) are diagrams illustrating modified examples of the method of switching the flow channel switching member according to Embodiment 1 of the present invention.

FIGS. 14(a) and 14(b) are diagrams illustrating modified examples of the method of switching the flow channel switching member according to Embodiment 1 of the present invention.

FIGS. 15(a) and 15(b) are diagrams illustrating modified examples of the method of switching the flow channel switching member according to Embodiment 1 of the present invention.

FIGS. 16(a) and 16(b) are diagrams illustrating modified examples of the method of switching the flow channel switching member according to Embodiment 1 of the present invention.

FIG. 17 is a modified example of the overview of the cell culture apparatus according to Embodiment 1 of the present invention.

FIGS. 18(a) to 18(c) are diagrams illustrating an example and modified examples of a structure of a flow channel switching mechanism according to Embodiment 2 of the present invention.

FIG. 19 is a diagram illustrating a modified example of the structure of the flow channel switching mechanism according to Embodiment 2 of the present invention.

FIGS. 20(a) and 20(b) are diagrams illustrating examples of a configuration of an integrated flow channel which includes a flow channel switching mechanism according to Embodiment 3 of the present invention.

FIGS. 21(a) and 21(b) are diagrams illustrating modified examples of the configuration of the integrated flow channel which includes the flow channel switching mechanism according to Embodiment 3 of the present invention.

FIGS. 22(a) to 22(c) are diagrams illustrating modified examples of the configuration of the integrated flow channel which includes the flow channel switching mechanism according to Embodiment 3 of the present invention.

FIG. 23 is a diagram illustrating a modified example of the configuration of the integrated flow channel which includes the flow channel switching mechanism according to Embodiment 3 of the present invention.

FIG. 24 is a diagram illustrating a modified example of the configuration of the integrated flow channel which includes the flow channel switching mechanism according to Embodiment 3 of the present invention.

FIGS. 25(a) and 25(b) are diagrams illustrating modified examples of the configuration of the integrated flow channel which includes the flow channel switching mechanism according to Embodiment 3 of the present invention.

FIG. 26 is a diagram illustrating a modified example of the configuration of the integrated flow channel which includes the flow channel switching mechanism according to Embodiment 3 of the present invention.

FIGS. 27(a) and 27(b) are diagrams illustrating modified examples of the configuration of the integrated flow channel which includes the flow channel switching mechanism according to Embodiment 3 of the present invention.

FIG. 28 is a diagram illustrating an example of an overview of cell culture using a cell culture apparatus according to Embodiment 4 of the present invention.

FIGS. 29(a) and 29(b) are diagrams illustrating examples of a control time chart in the cell culture using the cell culture apparatus according to Embodiment 4 of the present invention.

FIG. 30 is a diagram illustrating a modified example of the control time chart in the cell culture using the cell culture apparatus according to Embodiment 4 of the present invention.

FIGS. 31(a) and 31(b) are diagrams illustrating examples of a state of liquid supply in the cell culture using the cell culture apparatus according to Embodiment 4 of the present invention.

FIGS. 32(a) and 32(b) are diagrams illustrating examples of drive control of a camera and a culture container in the cell culture using the cell culture apparatus according to Embodiment 4 of the present invention.

### DESCRIPTION OF EMBODIMENTS

In the embodiments below, although a description will be given by dividing the embodiment into a plurality of sections or embodiments if necessary for convenience, these are not irrelevant to each other excepting the case that is particularly demonstrated, but are in a relationship in which one is a modified example of part or all of the other, a detailed description, a supplementary description, or the like. In addition, in the embodiments below, when the number of elements and the like (including the number, a numeric value, a quantity, a range, and the like) are stated, the embodiment is not limited to a particular number excepting the case that is particularly demonstrated or a case in which the embodiment is clearly limited, in principle, to the particular number, and the number may be more than or less than the particular number.

Further, in the embodiments below, it is obvious that the constituent components (including component steps and the like) are not necessarily required, excepting the case that is particularly demonstrated or a case in which the components are clearly required in principle. Similarly, in the embodiments below, when shapes, positional relationships and the like of the constituent components are stated, it is assumed that those substantially approximate to or analogous to the shapes and the like are included excepting the case that is particularly demonstrated or a case in which the components are obviously inappropriate in principle. This also applies to the numeric value and the range described above.

### Overview of Embodiment

First, an overview of an embodiment will be described. The overview of the embodiment will be described by providing reference numeral or the like of constituent components corresponding to the embodiment in parentheses, for example.

A representative cell culture apparatus of the present embodiment is a cell culture apparatus in which a plurality of sealed culture containers (culture containers 1, 35, 41 and 44) having fluid introduction ports and discharge ports are provided, and the plurality of culture containers are connected in parallel, forming a single closed culture system. The cell culture apparatus has a single flow channel switching mechanism (flow channel switching mechanism 8 or 19) for switching a plurality of flow channels connected to the plurality of culture containers.

More preferably, the flow channel switching mechanism in the cell culture apparatus makes a flow channel resistance of one individual flow channel from among the individual flow channels branching and being connected to the culture containers less than the flow channel resistance of the remainder of the individual flow channels.

More preferably, the flow channel switching mechanism in the cell culture apparatus forms an individual flow channel having a small flow channel resistance by placing a single flow channel switching member (flow channel switching member 9, 12, 13, 29, 37 or 39) in the closed culture system and changing at least one of an orientation and a position of the flow channel switching member.

Hereinafter, a detailed description will be given regarding the respective embodiments based on the overview of the embodiment described above with reference to the drawings. Incidentally, the same reference numerals will be attached to members having the same function, in principle, in the entire drawing for describing the respective embodiments, and the repetitive description thereof will be omitted. In addition, the description of the same or similar portions will not be repeated in principle unless particularly required in the respective embodiments.

In addition, there is a case in which hatching is not illustrated even in a cross-sectional view in order for the viewability of the drawing in some drawings to be used in the respective embodiments. In addition, there is a case in which hatching is illustrated even in a plan view in order for the viewability of the drawing.

### Embodiment 1

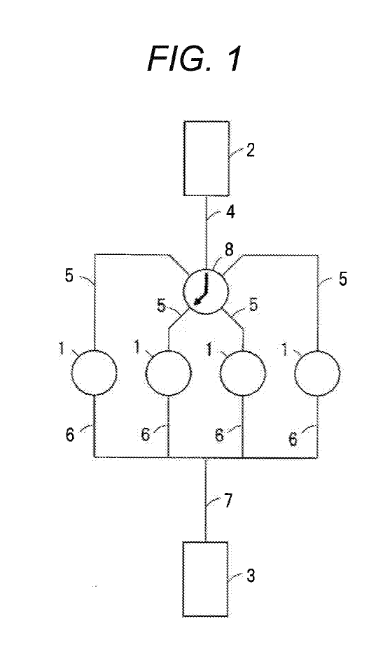

A cell culture apparatus according to the present embodiment will be described with reference to FIGS. 1 to 17. The cell culture apparatus according to the present embodiment is an example of a cell culture apparatus in which a plurality of sealed culture containers are connected in parallel, thereby forming a single closed culture system.

### <Cell Culture Apparatus>

FIG. 1 is a diagram illustrating an example of an overview of the cell culture apparatus according to the present embodiment.

The cell culture apparatus according to the present embodiment includes a culture container 1, a supply bag 2 in which a culture medium or the like is housed, a collection bag 3 which collects the used culture medium or the like, and a flow channel switching mechanism 8. The culture container 1, the supply bag 2, and the collection bag 3 are connected via flow channels in this cell culture apparatus. A plurality of (four in the example of FIG. 1) the culture containers 1 are provided to branch from common flow channels (a common flow channel 4 on an upstream side and a common flow channel 7 on a downstream side) using branching flow channels (branching flow channels 5 on the upstream side and branching flow channels 6 on the downstream side) to the respective culture containers 1. The flow channel switching mechanism 8 is provided in a branching portion from the common flow channel 4 on the upstream side into the branching flow channels 5 on the upstream side. In this manner, the flow channel switching mechanism 8 is arranged in the branching portion between the common flow channel 4 on the upstream side and the branching flow channels 5 on the upstream side.

Since the cell culture apparatus according to the present embodiment is the closed culture system, it is necessary to apply a driving force of a liquid from the outside of system. An example of the means for applying the driving force is a peristaltic pump that supplies the liquid by squeezing an elastic tube from an outer side. A least a part of the common flow channel may have the elasticity so as to enable the liquid supply using the peristaltic pump. The peristaltic pump may be installed in the common flow channel 4 on the upstream side or in the common flow channel 7 on the downstream side. In addition, the peristaltic pump may be installed in both the common flow channel 4 on the upstream side and the common flow channel 7 on the downstream side.

When the peristaltic pump (not illustrated) is driven, the liquid inside the supply bag 2 is transferred to the culture container 1. At the time, the liquid is transferred while changing the culture containers 1 by a switching operation of the flow channel switching mechanism 8. The liquid which has been originally contained in the culture container 1 is sent to be pushed out to the collection bag 3.

### <Flow Channel Switching Mechanism>

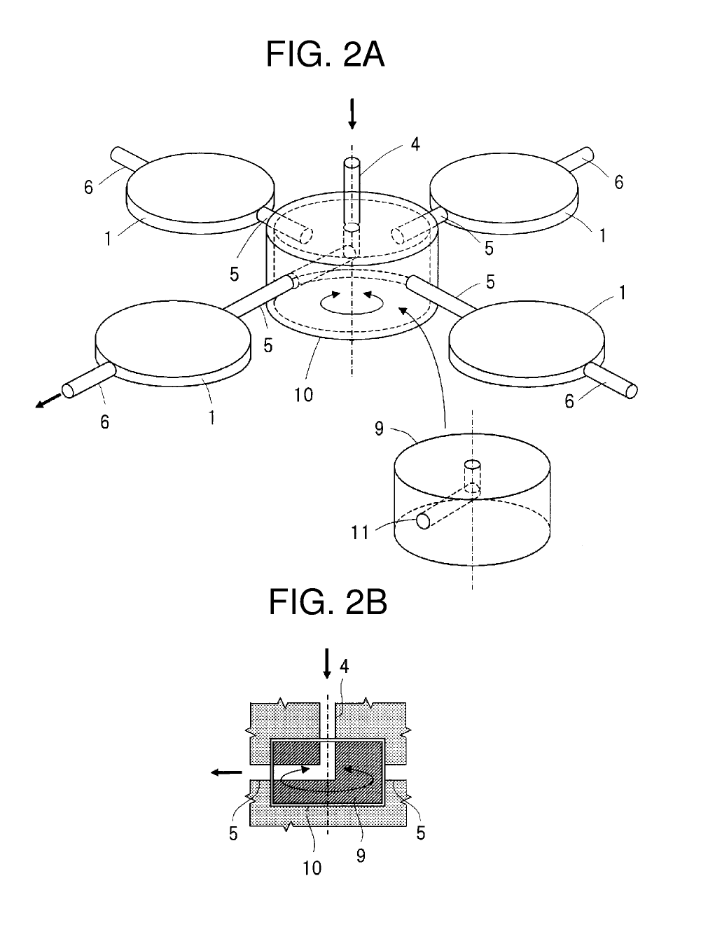

FIGS. 2(a) and 2(b) are diagrams illustrating an example of a structure of the flow channel switching mechanism described above. In FIGS. 2(a) and 2(b), FIG. 2(a) is a cubic diagram of the flow channel switching mechanism including the plurality of culture containers, and FIG. 2(b) is a side sectional view of the flow channel switching mechanism.

The flow channel switching mechanism 8 includes a flow channel switching member 9 and a storage compartment 10 in which low channel switching member 9 is stored. The common flow channel 4 on the upstream side and the plurality of (four in the examples of FIGS. 2(a) and 2(b)) the branching flow channels 5 on the upstream side are connected together with the storage compartment 10. When all the connected flow channels are blocked, the storage compartment 10 forms a sealed space. The flow channel switching member 9 is provided in the storage compartment 10, and has a single connecting flow channel 11 therein. The connecting flow channel 11 can face one of the plurality of branching flow channels 5 on the upstream side by changing an orientation of the flow channel switching member 9, and at this time, a single flow channel is formed from the common flow channel 4 on the upstream side to an arbitrary one of the branching flow channels 5 on the upstream side via the flow channel switching member 9.

The flow channel switching member 9 has a cylindrical shape in FIGS. 2(a) and 2(b). A cylindrical hole extending from a center of an upper surface downward intersects with a cylindrical side hole, thereby forming a single flow channel in the flow channel switching member 9. The storage compartment 10 has a tubular shape such that the cylindrical member is fit therein. The common flow channel 4 on the upstream side is connected together at a position corresponding to the upper-surface hole of the flow channel switching member 9 on the central axis thereof. In addition, the plurality of branching flow channels 5 on the upstream side are connected together at a height corresponding to the side hole of the flow channel switching member 9 on a side surface of the storage compartment 10. When the flow channel switching member 9 is rotated around the central axis, it is possible to allow the connecting flow channel 11 to face an arbitrary one of the branching flow channels 5 on the upstream side. In this manner, the flow channel switching mechanism 8 performs the switching of the flow channels inside the closed system.

Incidentally, the up and down may be reversed in FIGS. 2(a) and 2(b).

Even if a part of the liquid flows out to an undesired branch in the closed culture system that switchably supplies the liquid to the plurality of culture containers 1 being connected in parallel, such a liquid is originally planned to flow, and thus, there is no influence on the culture quality. Thus, the branch for which the liquid supply is not desired is not necessarily closed completely, but may be provided with a difference in flow channel resistance against a branch for which the liquid supply is desired to make the liquid hardly flow thereto in terms of an object of homogeneously supplying the liquid to the respective culture containers 1.

Therefore, a size of the flow channel switching member 9 may be a size which is slightly smaller than the storage compartment 10 so as to enter the storage compartment 10 with a gap. The supplied liquid can flow out to the undesired branch from the slight gap between the flow channel switching member 9 and the storage compartment 10, but there is no problem on the quality as described above even if the liquid has flown out. In addition, it is not preferable to raise liquid supply speed from a point of view of a fixing property of a cell in fact, and the above-described flowing out rarely occurs when the flow channel resistance is large to some extent since the liquid supply pressure is not high.

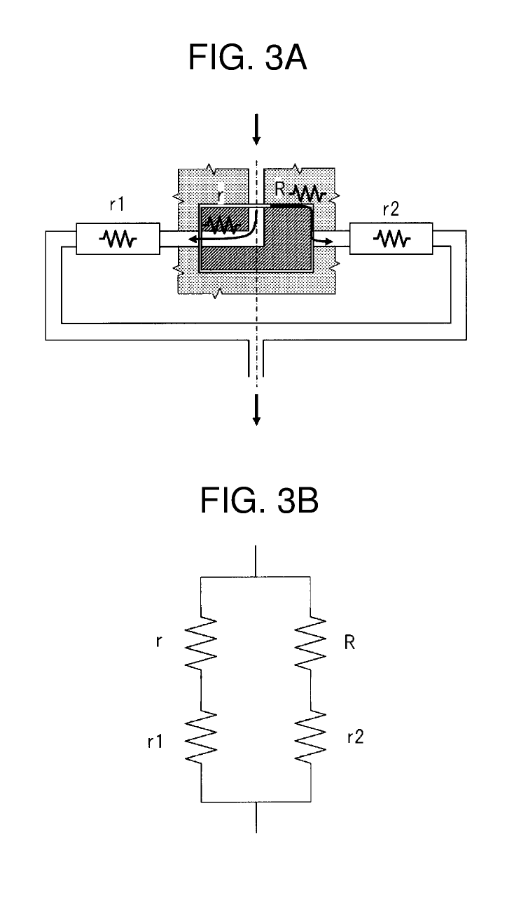

The flow channel switching mechanism 8 of the present embodiment is schematically drawn like FIGS. 3(a) and 3(b). FIGS. 3(a) and 3(b) are diagrams illustrating examples of the flow channel resistance of the flow channel switching mechanism. In FIGS. 3(a) and 3(b), FIG. 3(a) is a schematic view of the flow channel resistance of the flow channel switching mechanism, and FIG. 3(b) is an equivalent circuit diagram. Flow channel resistances after branching of the two branching flow channels are set to r1 and r2 (here, r1≈r2 since it is desirable that the flow channel resistances after branching be even), a flow channel resistance of the connecting flow channel 11 in the flow channel switching member 9 is set to r, and a flow channel resistance of a path formed by the slight gap between the flow channel switching member 9 and the storage compartment 10 is set to R.

(r1+r)<<(r2+R) Formula 1

When the relation of Formula 1 is established, the liquid rarely flows to the R side. The above-described Formula 1 is satisfied when r1 and r2 are set to be sufficiently smaller than R, and r<<R in the flow channel switching mechanism 8.

The following effect is obtained using a flow channel switching method according to the flow channel switching mechanism 8 of the present embodiment.

First, it is possible to lower a manufacturing cost of the flow channel switching mechanism 8. A high dimensional accuracy is required for the fitting of the flow channel switching member 9 and the storage compartment 10 in order to eliminate or infinitely suppress the flowing out of the supplied liquid to the branch other than the desired one. It is necessary to perform machining in some cases, thereby causing extremely high cost. The closed culture system including the flow channel switching mechanism 8 according to the present embodiment may be reused after performing sterilizing treatment for each culture, but may be preferably discarded after each culture in order to lower a risk of contamination. Accordingly, the manufacturing cost is extremely important. It is possible to loosen the fitting dimension in a case in which the slight flowing out of the supplied liquid is allowable as in the present embodiment. When a level of molding dimensional accuracy of a general molded article may be enough, the significant cost reduction is possible.

Another effect is that a force for switching the orientation of the flow channel switching member 9 is not required much. The flow channel switching member 9 and the storage compartment 10 are fitted with each other with the gap, and do not strongly contact each other in the flow channel switching method of the present embodiment. Thus, it is not necessary to exert a large force in order to change the orientation of the flow channel switching member 9. Accordingly, it is also possible to provide simple mechanism and structure for switching the flow channel switching member 9.

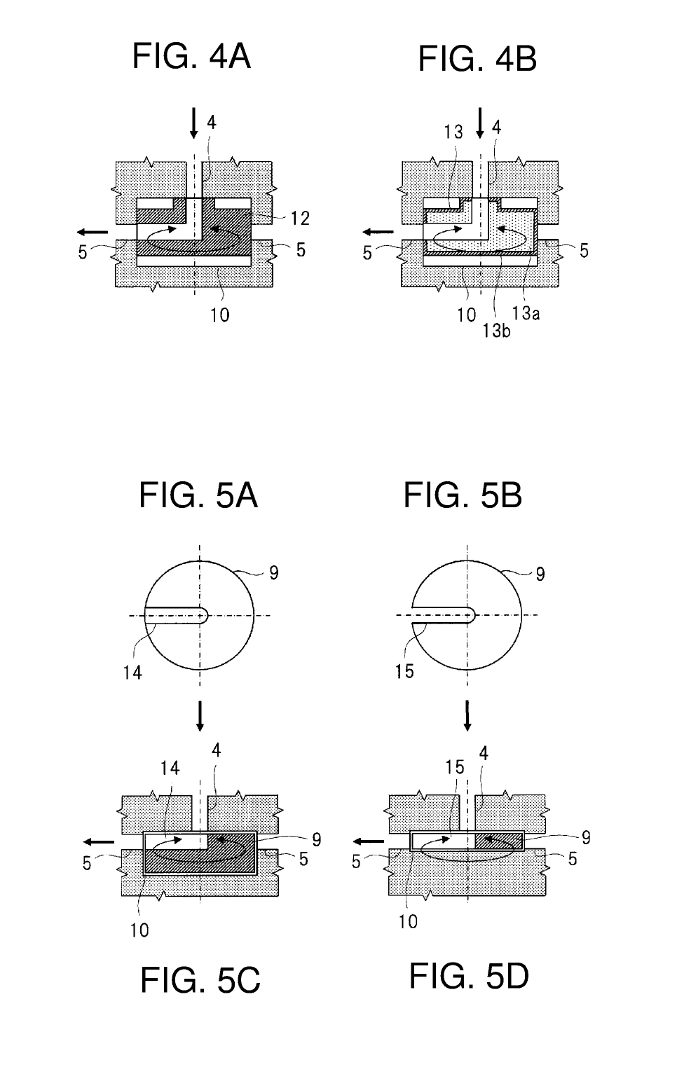

Hereinafter, a description will be given regarding modified examples of the structure of the flow channel switching mechanism in order with reference to FIGS. 4(a) to 11. Each of FIGS. 4(a) to 11 is a diagram illustrating the modified example of the structure of the flow channel switching mechanism.

The flowing out of the supplied liquid other than the desired one in the flow channel switching mechanism is wasteful although is not problematic in terms of the culture quality, it is desirable to tighten the fitting dimension between the flow channel switching member 9 and the storage compartment 10 as much as possible. Alternatively, a size of the flow channel switching member 9 may be set to be larger than the storage compartment 10 using a material with a low Young's modulus. An example thereof is illustrated in FIG. 4(a). A flow channel switching member 12 is made of rubber, and is stored in the state of being contracted to be in close contact along with a size of the storage compartment 10 in FIG. 4(a). Since the branching flow channel other than the desired one is sealed by the rubber, the undesired flowing out does not occur.

When the flow channel switching member 12 is in strong contacts the storage compartment 10, it is necessary to exert a large force to change an orientation of the flow channel switching member 12, and thus, it is preferable if there is a scheme to reduce the force. For example, it is preferable to reduce the contact surface as much as possible in order to decrease friction on the sliding surface. A material with high lubricity like Teflon may be used.

The material having the low Young's modulus may be applied to either side or both sides of the flow channel switching member and the storage compartment. Alternatively, the material may be applied partially not to the entire member. An example thereof is illustrated in FIG. 4 (b). The flow channel switching member 13 has an inner member 13a made of a material for a general structure being coated with rubber 13b in FIG. 4 (b), is in close contact with the storage compartment 10 at the rubber 13b portion, and suppresses the undesired flowing out.

The connecting flow channel to be formed in the flow channel switching member 9 is not limited to a tube structure, but may be a structure like a groove. Examples thereof are illustrated in FIGS. 5(a) to 5(d). In FIGS. 5(a) to 5(d), FIG. 5(a) is a top view of the flow channel switching member which includes a connecting flow channel having the groove structure, and FIG. 5 (b) is a side sectional view also including a storage unit. A groove 14 is formed on the upper surface of the cylindrical flow channel switching member 9 including the vicinity of center, and this portion becomes the connecting flow channel. When the connecting flow channel has such a shape, it is possible to achieve miniaturization of the flow channel switching mechanism. In addition, the groove structure of the flow channel switching member 9 may be formed like a groove 15 to notch the entire flow channel switching member 9 as illustrated in FIGS. 5(c) and 5(d). In FIGS. 5(a) to 5(d), FIG. 5(c) is a top view of the flow channel switching member which includes the groove notching the entire member as a connecting flow channel, and FIG. 5(d) is a side sectional view also including the storage unit.

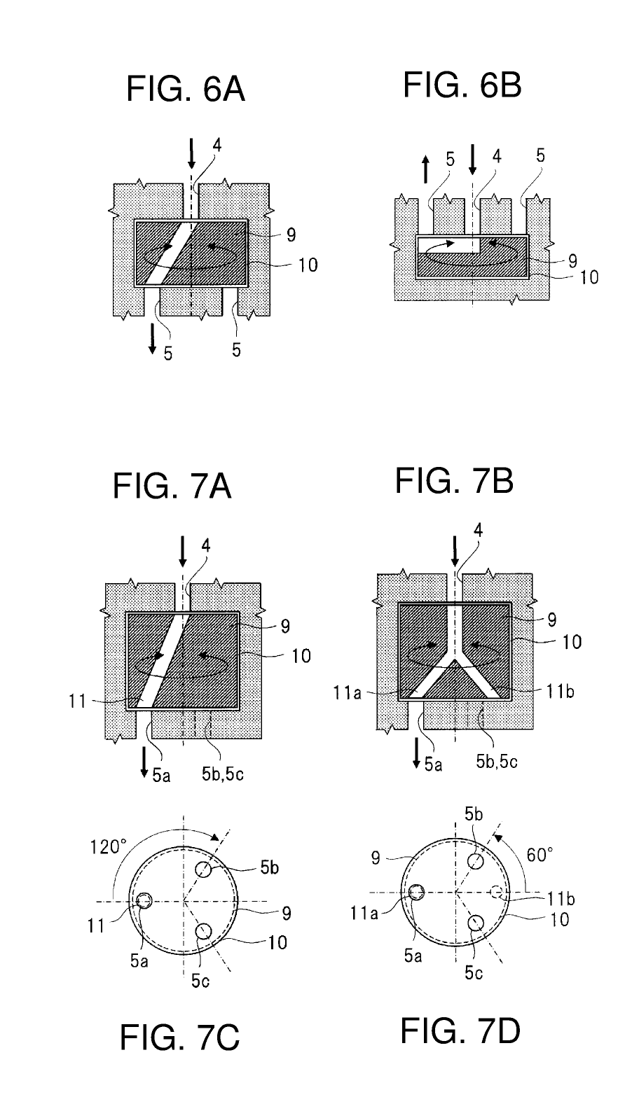

In addition, the branching flow channel 5 to be connected together can be provided on a lower surface as illustrated in FIG. 6(a) instead of the side surface of the storage compartment 10, and further, can also be provided on the same surface with the common flow channel 4 as illustrated in FIG. 6(b).

It may be enough when the connecting flow channel of the flow channel switching member forms the single path having the low the flow channel resistance at the time of supplying the liquid, and the number thereof is not limited to one but may be plural. Examples thereof are illustrated in FIGS. 7(a) to 7(d). In FIGS. 7(a) to 7(d), three branching flow channels 5a, 5b and 5c are connected together with the storage compartment 10 of the flow channel switching mechanism. In a case in which the three branching flow channels 5a, 5b and 5c are connected together with the storage compartment 10 with an interval of 120°, for example, as illustrated in FIGS. 7(a) and 7(b), a rotation by 120° is required for performing switching to the next branching flow channel, and a rotation by 180°, at maximum, is required to rotate the connecting flow channel 11 to face a desired branching flow channel from an arbitrary position when the number of the connecting flow channel 11 is one. Meanwhile, when two branching connecting flow channels (connecting flow channels 11a and 11b) are provided as illustrated in FIGS. 7(c) and 7(d), it is possible to perform switching to the next branching flow channel by a rotation by 60°, and to rotate the connecting flow channel 11 to face a desired branching flow channel from an arbitrary position by a rotation by 90° at maximum. In this manner, there is an effect of allowing the decrease in the amount of rotational movement required for the switching of the flow channels by providing the plurality of the connecting flow channels 11a and 11b.

It is advantageous when the flow channel switching member, which is switched by the rotation, has the connecting flow channel such that an end thereof is positioned at a central axis of rotation. When the common flow channel is arranged on the central axis of rotation, the common flow channel and the connecting flow channel necessarily face each other even when the rotation of the flow channel switching member is arbitrary.

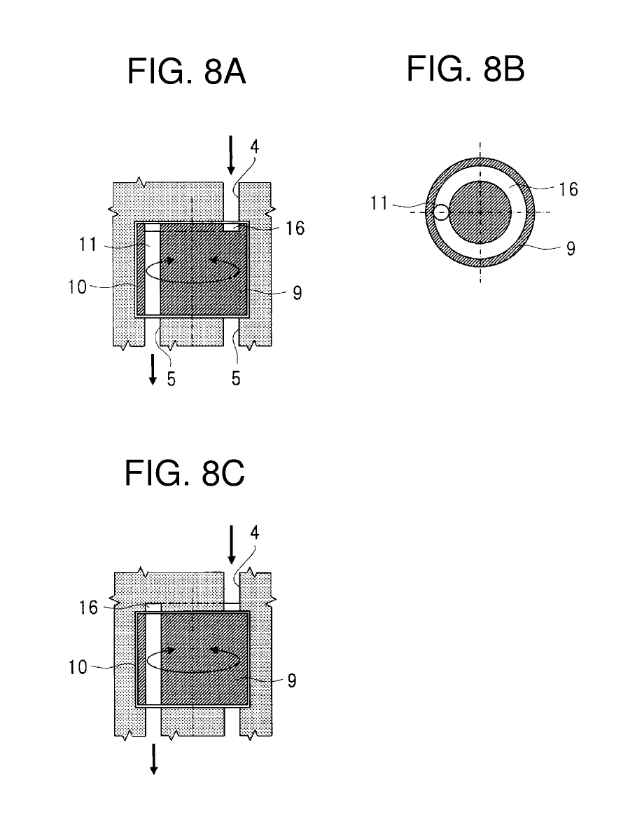

Meanwhile, the flow channel switching member may have the connecting flow channel of which one end is not intentionally positioned at the central axis of rotation. Examples thereof are illustrated in FIGS. 8(a) to 8(c). In FIG. 8(a), the connecting flow channel 11 of the flow channel switching member 9 is at a position which is eccentric from the central axis of rotation. The common flow channel 4 is also at the same eccentric position, but is difficult to communicate depending on a rotation angle of the flow channel switching member 9 in such a state. Therefore, an annular groove 16 is provided in the flow channel switching member 9 to allow communication to the branching flow channel 5 through the groove 16. FIG. 8(b) is a top view of the flow channel switching member 9. The annular groove 16 may be provided not on the flow channel switching member 9 side, but on the storage compartment 10 side as illustrated in FIG. 8(c).

It is possible to obtain the following effect with the above-described structure.

In a case in which one end of the connecting flow channel is positioned at the central axis of rotation, the number of the connecting flow channels is two at most on the upper surface and the lower surface of the cylinder. However, it is possible to provide an innumerable connecting flow channels in principle if one end of the connecting flow channel is not necessarily provided at the central axis of rotation.

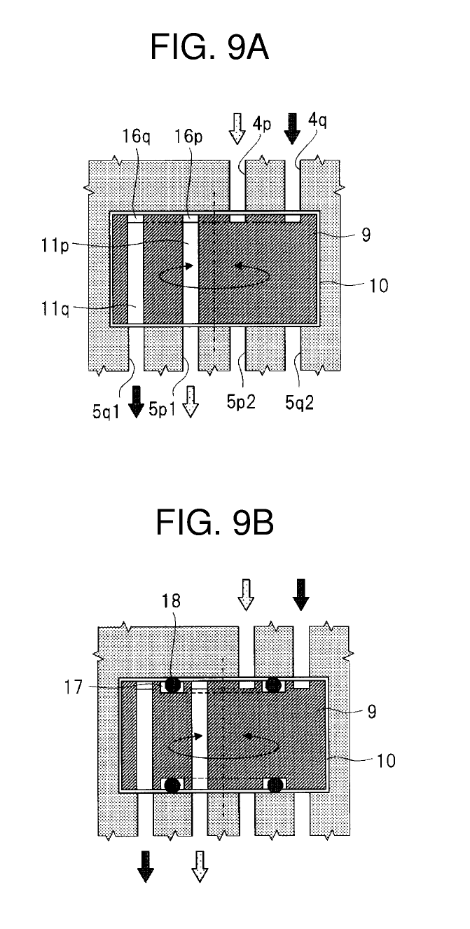

When a plurality of connecting flow channels are provided, it is possible to perform the switching of the flow channels of the plurality of common flow channel using the single flow channel switching member 9. Examples thereof are illustrated in FIGS. 9(a) and 9(b). FIGS. 9(a) and 9(b) are examples in which the switching of the flow channels is performed with respect to two common flow channels using a single flow channel switching member. In FIG. 9(a), two connecting flow channels 11p and 11q are provided in the flow channel switching member 9 and have annular grooves 16p and 16q, respectively. Common flow channels 4p and 4q are connected to the storage compartment 10 and can be switched, respectively, to a branching flow channel 5p1 or 5p2, and a branching flow channel 5q1 or 5q2. An annular groove 17 is additionally provided between the two annular grooves to be partitioned by an O-ring 18 as illustrated in FIG. 9(b) such that two systems of liquids are not mixed.

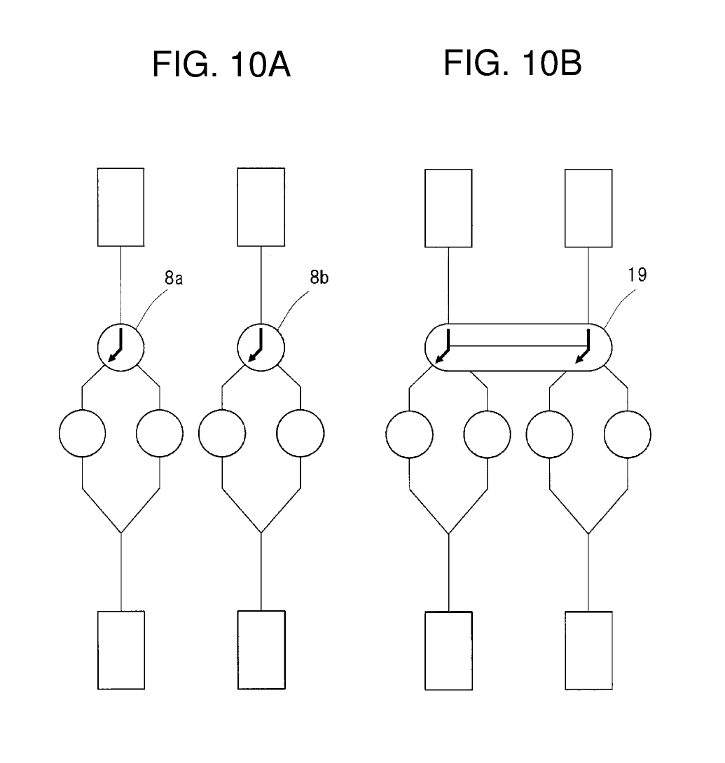

When the above-described flow channel switching member is provided, two flow channel switching mechanisms 8a and 8b, illustrated in FIG. 10(a), can be put together as the single flow channel switching mechanism 19 illustrated in FIG. 10(b).

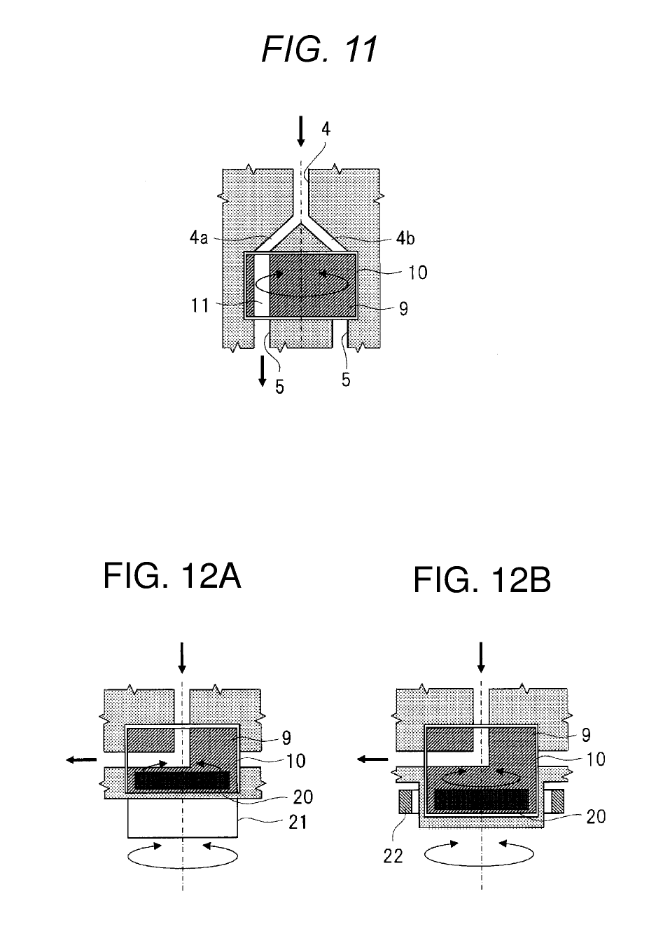

FIG. 11 illustrates an alternative of the annular groove of FIG. 8(c) in which the common flow channel branches before entering the storage compartment. In this case, the entire flow channel to be connected together with the storage compartment 10 becomes the branching flow channel 5, and it can be similarly considered when parts (4a and 4b) to be connected together with the storage compartment 10 are regarded also as the common flow channels.

<Method of Switching Flow Channel Switching Member>

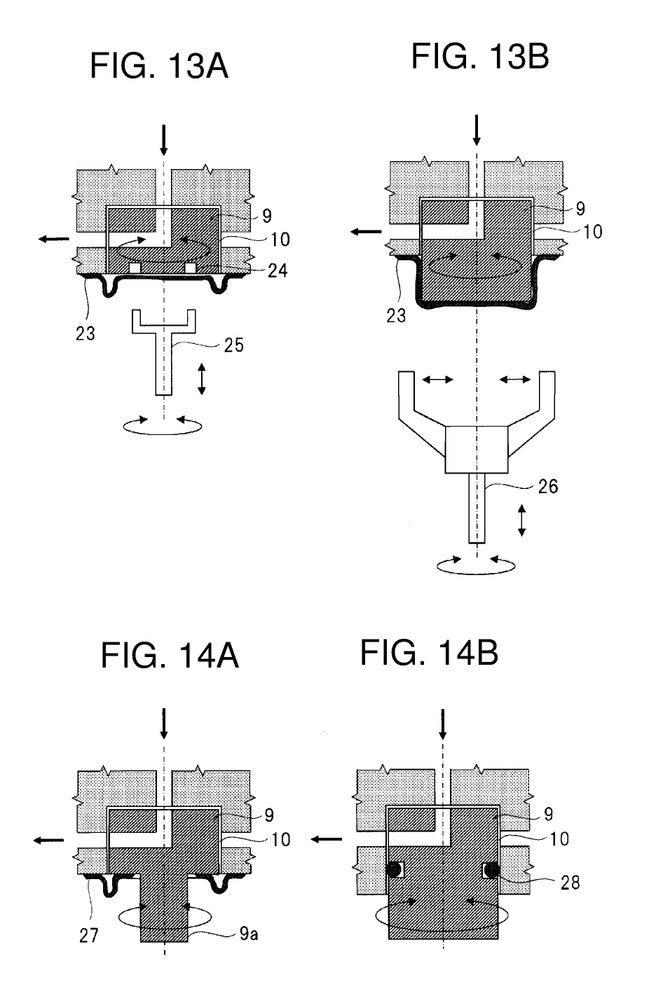

Next, a description will be given regarding a method of switching the orientation of the flow channel switching member. Hereinafter, a description will be given regarding examples and modified examples of the method of switching the flow channel switching member in order with reference to FIGS. 12 (a) to 16(b). FIGS. 12(a) and 12(b) are diagrams illustrating the examples of the method of switching the flow channel switching member. Further, each of FIGS. 13 (a) to 16(b) is a diagram illustrating the modified example of the method of switching the flow channel switching member.

Since the flow channel switching member is inside the closed system, it is difficult to directly contact the member for switching, and another scheme is required. One of the switching method uses a remotely applied force. Examples of the remotely applied force that can be used includes a magnetic force and gravity.

FIGS. 12(a) and 12(b) illustrate the method of switching the flow channel switching member using the magnetic force. The flow channel switching member is made of a magnetic material, but, alternatively, may have the magnetic material being buried therein. A magnetic field generation means is provided in addition to the storage compartment, and the orientation of the switching member is changed by varying a magnetic field. In FIG. 12(a), a magnet 20, which is a magnetic body, is buried in the flow channel switching member 9, and acts against another magnet 21 through a wall of the storage compartment 10, and the orientation of the flow channel switching member 9 is changed by rotating the magnet 21. When a magnet having a large retention force is used, the orientation is more easily defined. As illustrated in FIG. 12 (b), a magnet 22 having a torus shape may be rotated. The friction is scarcely generated between the flow channel switching member 9 and the storage compartment 10 from an orientation of a suction force in the above-described structure, and it is possible to perform the switching with a small force.

An electromagnet may be used as the magnetic field generation means.

The orientation of the force is a single direction and is not changed in the case of using the gravity, and thus, the switching is difficult as compared to the case of using the magnetic force, but it is possible to switch the flow channel by using a switching member of which the center of gravity is made eccentric and rotating the storage compartment side.

Next, a description will be given regarding a method of indirectly grabbing and switching the flow channel switching member. Examples thereof are illustrated in FIGS. 13(a) and 13(b). In FIG. 13(a), a part of the storage compartment 10 is opened, and a film 23 with elasticity or flexibility is bonded as a sealing member to block the opening portion. Although the closed system is maintained since the opening portion is blocked by the film 23, it is possible to grab the flow channel switching member 9 and transmit an external force via the film 23 since the film 23 is deformable.

It is preferable to provide a scheme that facilitates the grabbing in the flow channel switching member 9. For example, a concave structure 24 is provided in a part of the flow channel switching member 9, and the flow channel switching member 9 is grabbed by an external force transmission mechanism 25 having a counterpart convex structure via the film 23 as illustrated in FIG. 13(a).

A part of the flow channel switching member 9 may be formed to protrude from the storage compartment 10 as illustrated in FIG. 13(b) in order to facilitate the grabbing. The closed system is maintained by covering the storage compartment 10 including the protruding portion using the film 23. The external force transmission mechanism 26 serves a function of grabbing, rotating, and releasing the protruding portion of the flow channel switching member 9 via the film 23.

Although the film 23 has elasticity or flexibility, there is a limit in variable amount, and thus, a scheme is required to prevent the film 23 from being rotated more than necessary in the state of being grabbed. For example, there is a method of dividing the amount of rotation and changing the grabbing instead of rotating at a time. It is possible to repeat the rotation operation as deformation of the film is recovered by the elasticity or the deflection is recovered at the time of releasing the film for changing the grabbing. It is also effective to combine the above-described scheme with the scheme for reducing the amount of rotation at the time of the switching by providing the plurality of connecting flow channel s in the flow channel switching member as illustrated in FIGS. 7(a) to 7(d) described above.

As illustrated in FIGS. 14(a) and 14(b), it may be configured such that a part of the flow channel switching member 9 is exposed to the outside of the closed system to be switched by directly grabbing an exposed portion 9a. An arbitrary sealing means is provided at a boundary between the exposed portion 9a and an inner portion of the closed system to maintain the closed system. In FIG. 14(a), the film 27 having elasticity or flexibility is bonded to both the storage compartment 10 and the flow channel switching member 9 to perform the sealing. There is also a sealing method of using an O-ring 28 or oil seal as illustrated in FIG. 14(b).

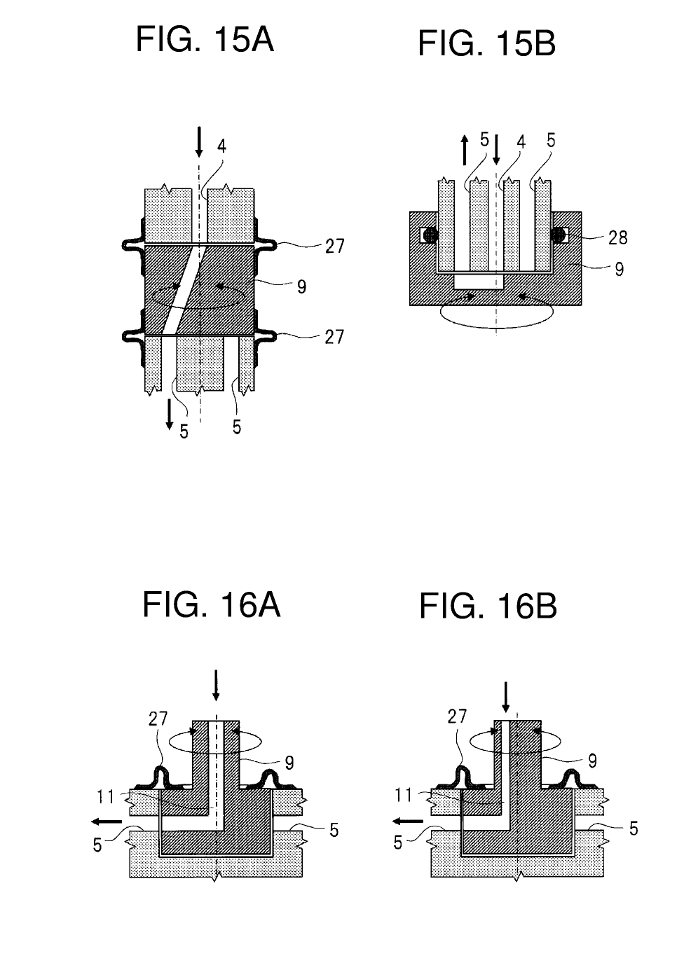

It may be enough when the flow channel switching member 9 has the connecting flow channel being arranged inside the closed system, and thus, it is not necessarily to accommodate the flow channel switching member 9 in the storage compartment 10. It may be configured such that the structure which is called the storage compartment is not provided as illustrated in FIG. 15(a), and the flow channel switching member 9 directly has the structure of the storage compartment as illustrated in FIG. 15(b). In FIG. 15(a), the flow channel switching member 9 has openings of the connecting flow channel on the upper surface and the lower surface, and a position corresponding the side surface is in an opened state, but is blocked by the film 27 having elasticity or flexibility. Accordingly, the closed system is maintained. In FIG. 15(b), the common flow channel 4 and the branching flow channel 5 are on the same surface, and the flow channel switching member 9 has a shape to cover such portions. A sealing means is provided so as to prevent the closed system from collapsing, and the O-ring 28 is used in FIG. 15(b).

As illustrated in FIGS. 16(a) and 16(b), a part of the flow channel switching member 9 is exposed to the outside of the closed system, and an end of a connecting flow channel may be formed out from the outside exposed portion thereof. The connecting flow channel serves a function also as the common flow channel, and can be directly connected with the supply bag. In FIG. 16(a), the connecting flow channel 11 serving the function also as the common flow channel is positioned at the central axis of rotation of the flow channel switching member 9. In addition, there is no need of positioning the connecting flow channel 11 serving the function also as the common flow channel at the central axis of rotation of the flow channel switching member 9 by providing the structure as illustrated in FIG. 16(b), it is advantageous to provide an innumerable connecting flow channels in principle.

### Modified Example of Cell Culture Apparatus

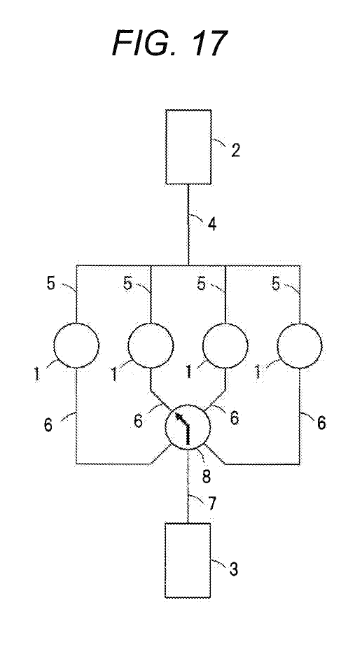

FIG. 17 is a diagram illustrating a modified example of the overview of the cell culture apparatus according to the present embodiment.

The flow channel switching mechanism 8 may be arranged on the downstream in the cell culture apparatus according to the present embodiment as illustrated in FIG. 17. In this case, the flow channel switching mechanism 8 is provided at a portion at which the branching flow channels 6 on the downstream side are merged to the common flow channel 7 on the downstream side.

In this manner, the flow channel switching mechanism 8 is arranged at the merging portion between the branching flow channels 6 on the downstream side and the common flow channel 7 on the downstream side.

In this flow channel switching mechanism 8, the liquid may enter the gap between the flow channel switching member and the storage compartment upon the structure, which becomes the dead volume. Such a liquid becomes wasteful if the flow channel switching mechanism 8 is at the upstream, but is not wasteful if the flow channel switching mechanism 8 is at the downstream which is a drainage side. In addition, when the flow channel switching mechanism 8 is arranged at the downstream, there is also an advantageous that the restriction with respect to the material of the member forming the flow channel switching mechanism 8 is mitigated. Since the liquid flows to the culture container 1 passing through the flow channel switching mechanism 8 in a case in which the flow channel switching mechanism 8 is arranged at the upstream, it is essential that the material of the component does not affect the culture, but the material is not limited as long as the flow channel switching mechanism 8 is at the downstream on the drainage side.

### Effect of Embodiment 1

As described above, it is possible to uniformly and homogeneously supply the liquid among the plurality of culture containers 1 while making the cost low, as the representative effects, by including the single flow channel switching mechanism 8 to switch the plurality of flow channels connected with the plurality of culture containers 1 according to the cell culture apparatus according to the present embodiment. As a result, it is possible to achieve homogenization in culture quality among the culture containers 1. In addition, it is possible to perform the cell culture simultaneously in the plurality of culture containers 1, and thus, it is possible to increase a yield by increasing the number of the culture containers 1. In addition, it is possible to change the number of the culture containers 1 or the like in accordance with a target yield, and thus, a flexible response with respect to the cell culture becomes possible. Other effects are as described as in the present embodiment.

Further, since it is possible to obtain a plurality of cultures, it is possible to provide one of the obtained cultures for inspection in addition to one for transplantation, for example, which is an additional effect. In the case of an invasive inspection such as a staining test, the inspection becomes a so-called destructive inspection, and thus, it is difficult to provide the inspected cell for transplantation. When a plurality of cells are cultured simultaneously as in the present embodiment, it is possible to allow a usage in which one of the cultures is taken out for inspection prior to transplantation, and the other cultures are used for transplantation after confirming that there is no problem in terms of quality.

### Embodiment 21

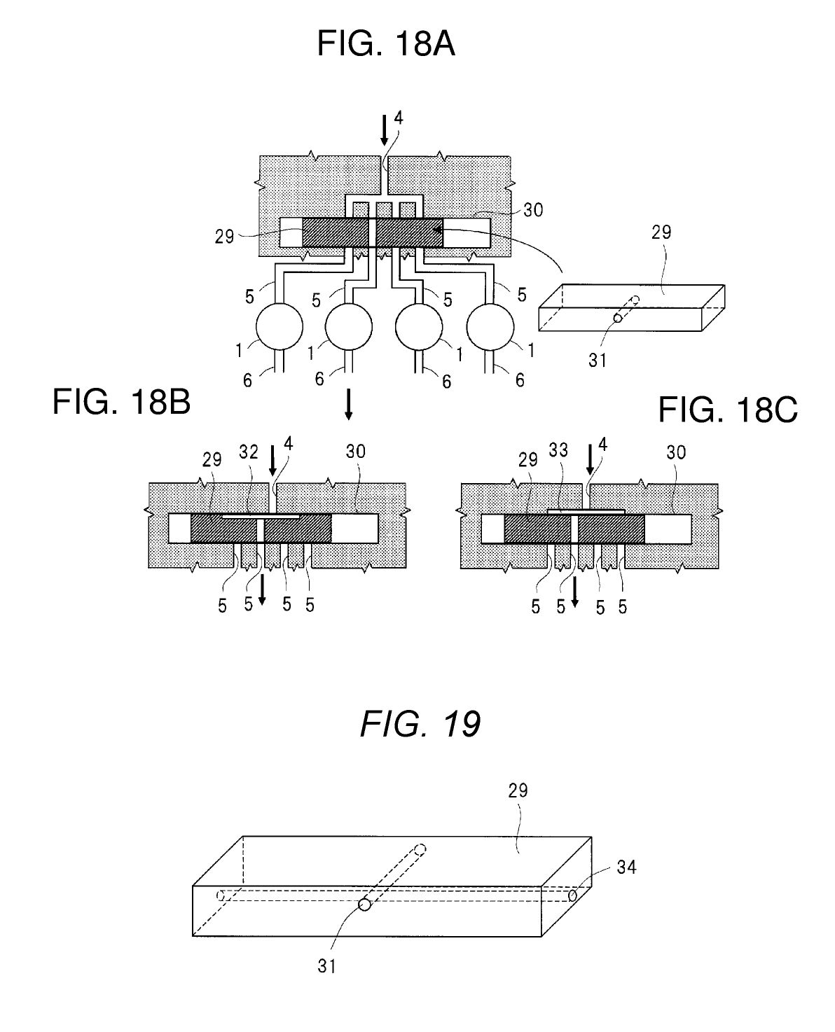

A cell culture apparatus according to the present embodiment will be described with reference to FIG. 18(a) to FIG. 19. FIGS. 18(a) to 18(c) are diagrams illustrating an example and modified examples of a structure of a flow channel switching mechanism. Further, FIG. 19 is a diagram illustrating a modified example of the structure of the flow channel switching mechanism.

A flow channel switching member may perform reciprocating motion such as linear motion or screwing motion without being limited to the rotational motion as in Embodiment 1 described above. A description will be given regarding an example of the flow channel switching member which performs the reciprocating motion in the present embodiment. A point that is different from Embodiment 1 described above will be mainly described in the present embodiment.

In FIG. 18(a), a flow channel switching member 29 is inside a storage compartment 30 and has a through-hole 31 as a connecting flow channel therein The upstream and the downstream of the branching flow channel 5 are connected together with the storage compartment 30 as a pair, and there is a space only for allowing the flow channel switching member 29 to move right and left. Any one of the branching flow channels 5 is selected by changing a position of the flow channel switching member 29.

It may be configured such that the connecting flow channel is formed in a shape of the through-hole 31 and a groove 32 are combined, and the common flow channel 4 is directly connected together with the storage compartment 30 as illustrated in FIG. 18 (b). In addition, a groove 33 may be provided in the storage compartment 30 side as illustrated in FIG. 18(c).

As illustrated in FIG. 19, a through-hole 34, which does not intersect the connecting flow channel, may be provided in the lateral direction in order to smoothly perform the right and left movement of the flow channel switching member 29 inside the storage compartment 30.

The flow channel switching member performing the rotational motion can be switched only by changing the orientation, and a range of motion of the flow channel switching member is not changed, and thus, it is possible to downsize the storage compartment. However, it is necessary to three-dimensionally arrange the common flow channel, the branching flow channel, and the connecting flow channel. On the contrary, it is possible to two-dimensionally arrange the above-described channels in the reciprocating motion equation as in the present embodiment, and thus, the latter configuration is suitable in a case in which it is desired to provide a thin flow channel switching mechanism.

Incidentally, a shape or the number of the connecting flow channels, a method of switching the flow channel switching member, and the like also can be considered similarly as those of the rotational motion equation even in the reciprocating motion equation.

As described above, it is possible to obtain a different effect as follows in addition to the same effects of Embodiment 1 described above according to the cell culture apparatus according to the present embodiment. For example, a representative effect is that it is possible to provide the thin flow channel switching mechanism since it is possible to two-dimensionally arrange the common flow channel, the branching flow channel, and the connecting flow channel by providing the flow channel switching member 29 which performs the reciprocating motion such as the linear motion and the screwing motion. Other effects are as described as in the present embodiment.

### Embodiment 3

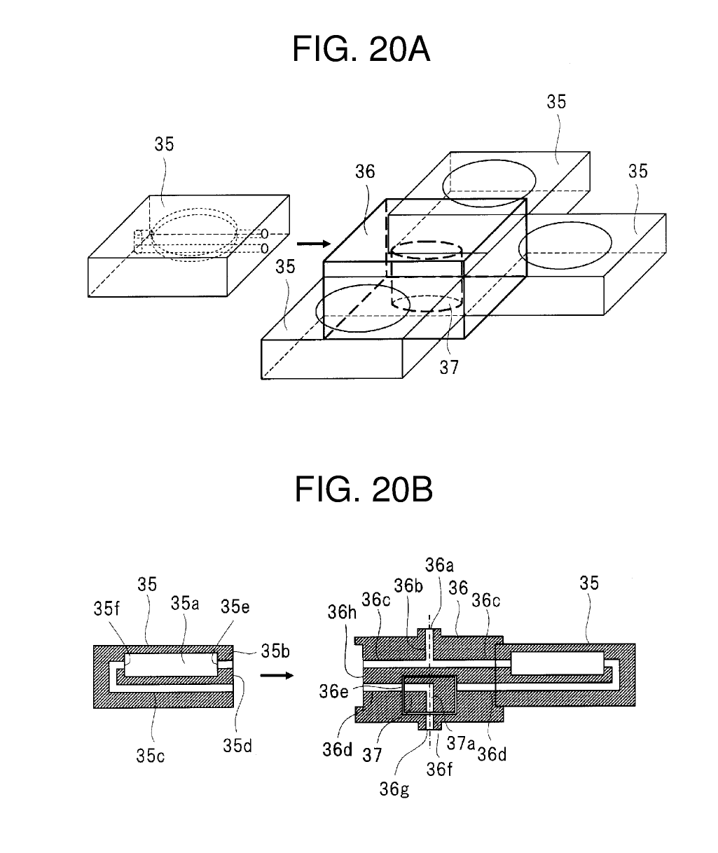

A description will be given regarding a cell culture apparatus according to the present embodiment with reference to FIGS. 20(a) to 27(b). FIGS. 20(a) and 20(b) are diagrams illustrating examples of a configuration of an integrated flow channel which includes a flow channel switching mechanism.

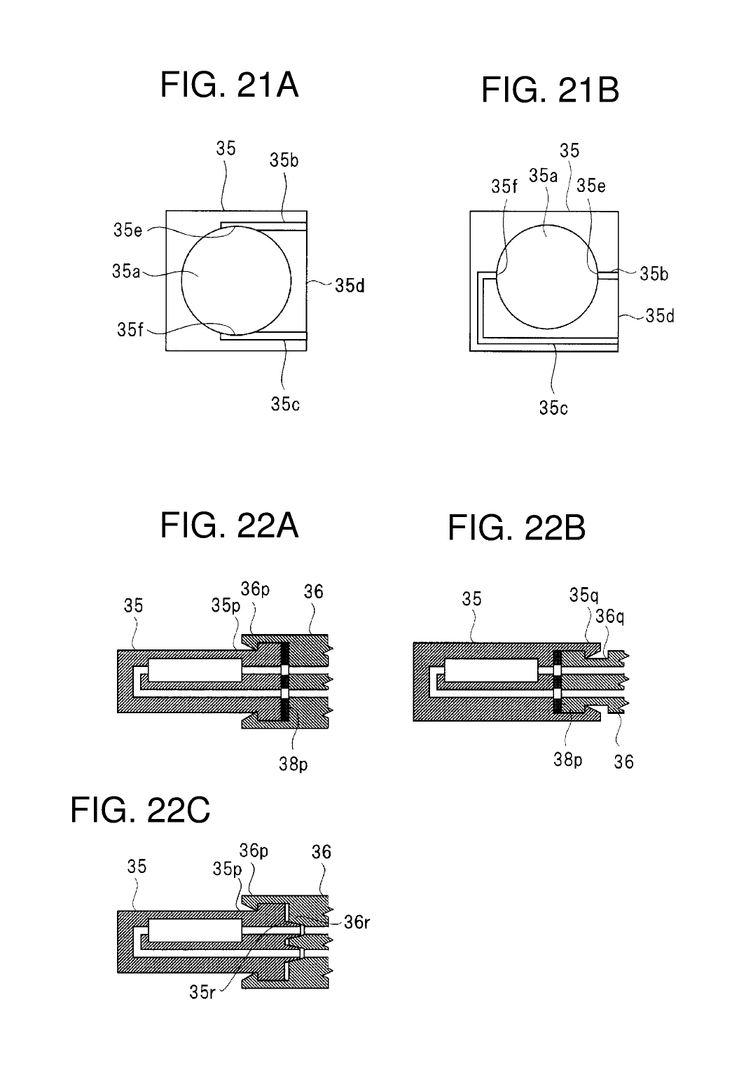

Further, each of FIGS. 21(a) to 27(b) is a diagram illustrating a modified example of the configuration of the integrated flow channel which includes the flow channel switching mechanism.

A description will be given regarding the example of the configuration of the integrated flow channel which includes the flow channel switching mechanism in the present embodiment. A point that is different from Embodiments 1 and 2 described above will be mainly described also in the present embodiment.

In a case in which the plurality of culture containers are connected in parallel, the corresponding number of branches are required, and the number of flow channels increases, which is complicated. When the individual flow channels are connected one by one, it takes a lot of man-hours, and further, there is a risk of erroneous piping. Therefore, it is preferable to collect these flow channels to form an integrated member (integrated flow channel member). Examples thereof are illustrated in FIGS. 20(a) and 20(b).

FIG. 20(a) is a cubic diagram in a state in which the culture container and the integrated flow channel member are combined including the flow channel switching member, and FIG. 20(b) is a side sectional view thereof. In the examples, the flow channel switching member is arranged on the downstream side.

A configuration which includes the culture container and the integrated flow channel member enclosing the flow channel switching member as illustrated in FIGS. 20(a) and 20(b) is referred to as a culture container set here.

A culture container 35 has a culture surface 35a, an inflow channel 35b, and a discharge flow channel 35c. An integrated flow channel member 36 has an inflow port 36a, an upstream common flow channel 36b, an upstream branching flow channel 36c, a downstream branching flow channel 36d, a storage compartment 36e serving as the flow channel switching member, a downstream common flow channel 36f, and a discharge port 36g. In addition, the culture container 35 and the integrated flow channel member 36 have a connection port 35d and ports 36h, respectively, to enable connection with each other. The culture container 35 has the single connection port 35d, and the integrated flow channel member 36 has a plurality of the ports 36h to enable connection with a plurality of containers. In addition, a flow channel switching member 37 is provided inside the storage compartment 36e.

The connection port 35d of the culture container 35 is linked with the inflow channel and the discharge flow channel, and the ports 36h of the integrated flow channel member 36 are linked with upper and lower branching flow channels. When the integrated flow channel member 36 and the culture container 35 are connected, the upstream branching flow channel 36c and the inflow channel 35b of the culture container 35, and the downstream branching flow channel 36d and the discharge flow channel 35c of the culture container 35 are caused to face each other. Desirably, each of the connection port 35d and the port 36h has one surface, and the above-flow channels are linked with the respective surfaces, and accordingly, the flow channels face each other by causing the surfaces match each other. In this manner, it is possible to attach and detach the culture container 35 from a single direction with respect to the integrated flow channel member 36, thereby facilitating the attachment and detachment. Each of the connection port 35d and the port 36h may have a plurality of surfaces in order for positioning or sealing, but an angle formed with respect to each normal line of surface need not exceed 180°. In this manner, the attachment and detachment from the single direction is possible even if the plurality of surfaces are provided although depending on the arrangement of surfaces.

An inlet 35e and an outlet 35f of the liquid in the culture surface 35a of the culture container 35 may be arranged to oppose each other with the culture surface 35a interposed therebetween such that the liquid supply spreads over the entire surface. Meanwhile, it is preferable to allow the inflow channel 35b and the discharge flow channel 35c to be linked with one surface of the connection port 35d as described above in order for easiness of connection with the integrated flow channel member 36, and thus, a scheme is required for the arrangement of flow channels inside the culture container 35. For example, the two inflow channel 35b and discharge flow channel 35c, which are parallel in a tangential direction of the circular culture surface 35a, are drawn out to be linked with the connection port 35d as illustrated in FIG. 21(a). Alternatively, any one of the inflow channel 35b and the discharge flow channel 35c, which are drawn out in the normal direction, may be folded back to be linked with the connection port 35d as illustrated in FIG. 21(b) (FIG. 21(b) is an example in which the discharge flow channel 35c is folded back).

When being installed, the culture container 35 may be pushed to be fit and fixed in the single direction using a snap-fit structure so as to enable the simple installation. Examples of the structure thereof are illustrated in FIGS. 22(a) to 22(c). In FIGS. 22(a) and 22(b), the integrated flow channel member 36 has a convex structure 36p, and the culture container 35 has a concave structure 35p. As illustrated in FIG. 22(c), the culture container 35 may have a convex structure 35q, and the integrated flow channel member 36 may have a concave structure 36q.

A scheme for preventing a leakage is provided between the ports 36h of the integrated flow channel member 36 and the connection port 35d of the culture container 35 using a seal such as a gasket 38p illustrated in FIGS. 22(a) and 22(c) and tapers 35r and 36r illustrated in FIG. 22(b).

It is also preferable to form the ports 36h of the integrated flow channel member 36 and the connection port 35d of the culture container 35 in asymmetric shapes so as not to be attached upside down.

When such shapes are formed, the closed culture system is formed only by connecting the culture container 35 to each of the ports 36h of the integrated flow channel member 36, and connecting the supply bag and the collection bag to the inflow port 36a and the discharge port 36g of the integrated flow channel member 36, respectively. As a result, it does not take the man-hours, and the erroneous piping with the culture container 35 hardly occurs. In addition, it is possible to lower the cost as the upper and lower common flow channels and branching flow channels are integrated.

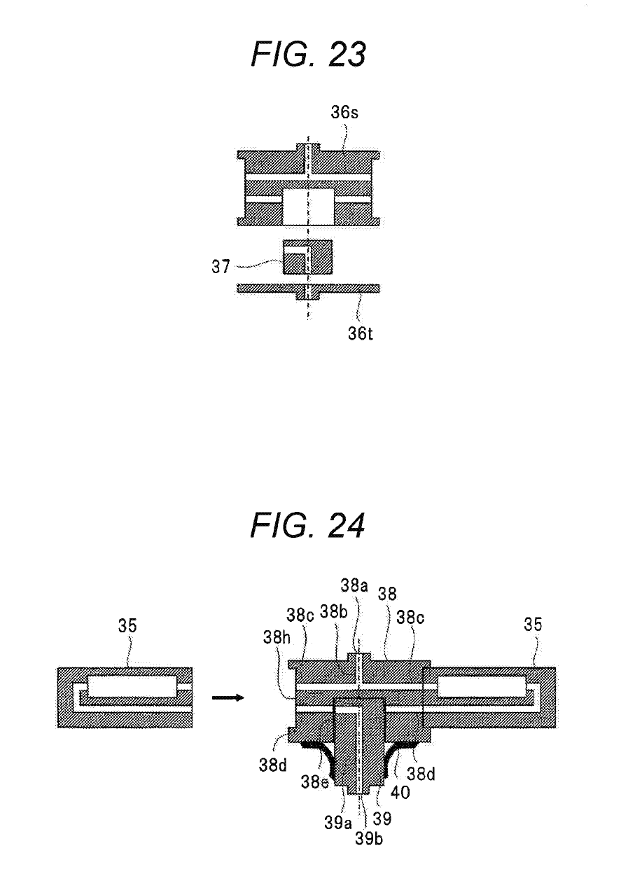

Incidentally, there is a method of dividing the integrated flow channel member into two divided members 36s and 36t, inserting the flow channel switching member 37 therebetween, and then joining the two divided members 36s and 36t using, for example, ultrasonic welding, as illustrated in FIG. 23, as a method of enclosing the flow channel switching member 37.

FIG. 24 is an example in which a part of the flow channel switching member is exposed outside the closed system so as to allow an external force to be directly transmitted to the flow channel switching member from the outside of the closed system. An integrated flow channel member 38 has an inflow port 38a, an upstream common flow channel 38b, an upstream branching flow channel 38c, a downstream branching flow channel 38d, a storage compartment 38e serving as the flow channel switching member, and a port 38h. The storage compartment 38e is opened and in which the flow channel switching member 39 is inserted. A film 40 with elasticity or flexibility is joined with each of the integrated flow channel member 38 and the flow channel switching member 39, and accordingly, the closed system is maintained. A sealing means using an O-ring or oil seal may be used as a means for maintaining the closed system. The flow channel switching member 39 has a connecting flow channel 39a, and an end thereof protrudes outside the closed system as a discharge port 39b. This connecting flow channel 39a serves a function also as the downstream common flow channel. The supply bag may be connected to the discharge port 39b of the flow channel switching member 39. The similar configuration as the example in which the flow channel switching member is enclosed may be applied regarding the other members.

Incidentally, it may be enough that the switching of the flow channels allows the flow channel switching member 39 and a desired branching flow channel inside the integrated flow channel member 38 to be aligned to face each other, and the drive target is not necessarily limited to the flow channel switching member. It may be configured such that the flow channel switching member is fixed, and the integrated flow channel member is driven.

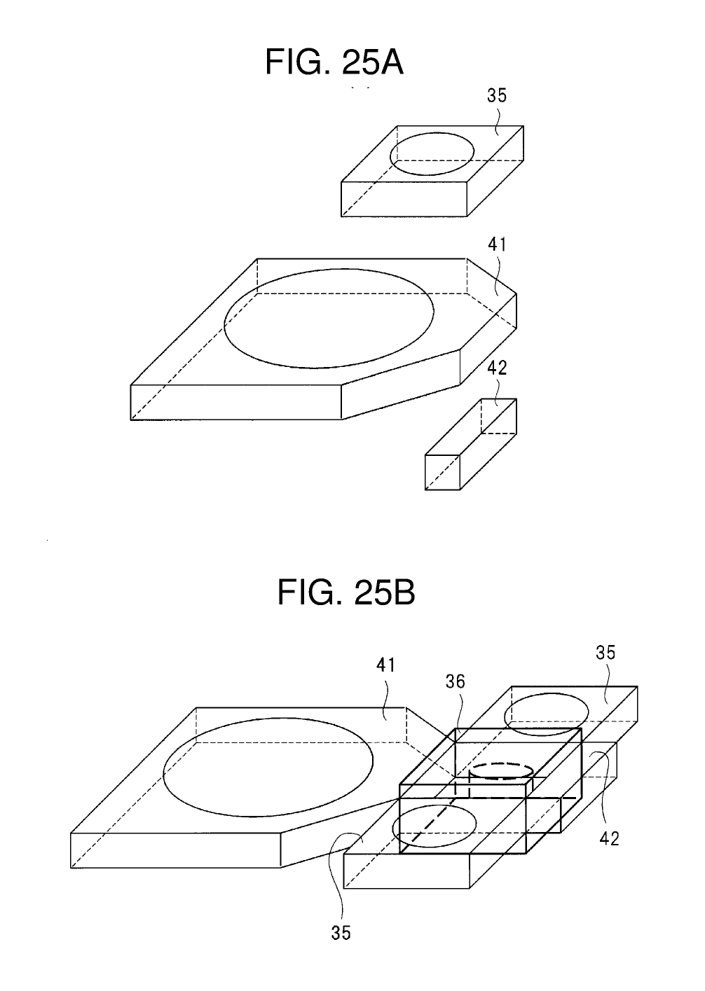

As illustrated in FIG. 25(a), culture containers of which sizes of culture surfaces are changed instead of changing shapes and sizes of connection ports may be used. The culture container 35 and a culture container 41 have different sizes of culture surfaces, but have the same shape and size of connection ports Thus, it is possible to use the integrated flow channel member without change even when the culture container is changed. In this manner, the culture containers 35 and 41 having different culture surfaces may be mixed.

In addition, a stopper 42, formed in accordance with the shape of the connection port may be provided. It may be enough if the stopper 42 has a function of sealing a liquid, and thus, it is possible to manufacture the stopper 42 with low cost. It may be configured such that a required number of the culture containers are connected to the integrated flow channel member, and the stopper 42 is used for the remaining port.

FIG. 25(b) illustrates a state in which culture containers 35 and 41, which have different sizes of culture surfaces, and a stopper 42 are connected to the integrated flow channel member 36. The flexible response becomes possible in accordance with a target culture.

The culture container can be detached. An application of detaching the culture container for inspection is considered. The stopper may be used after detaching the culture container, and it is preferable when there is a device which is capable of blocking each of the integrated flow channel member side and the culture container side to be aseptically separated at the time of detaching the culture container.

It is preferable that the culture container, the integrated flow channel member, and the flow channel switching member be resin-molded products in terms of cost. Polystyrene, polypropylene, polycarbonate or the like, which is a material to be used for a general culture container, may be used as the material thereof.

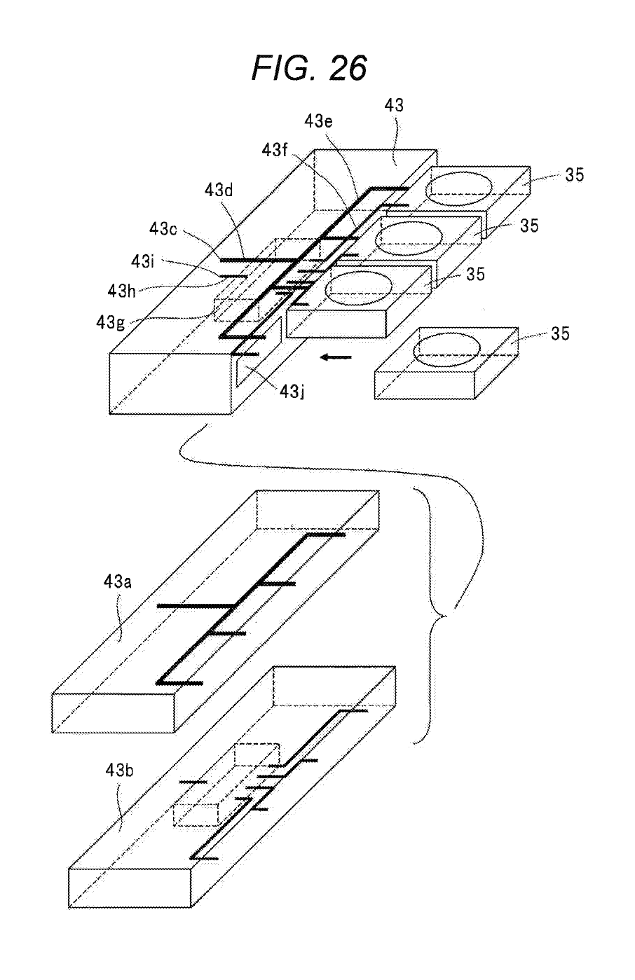

FIG. 26 is an example of the integrated flow channel member in a case in which the flow channel switching member performs the reciprocating motion. The flow channel switching member is arranged on the downstream side. An integrated flow channel member 43 is roughly divided into an upstream part 43a and a downstream part 43b, and has a structure in which the two parts are integrated. The integrated flow channel member 43 has an inflow port 43c, an upstream common flow channel 43d, an upstream branching flow channel 43e, a downstream branching flow channel 43f, a storage compartment 43g serving as the flow channel switching member, a downstream common flow channel 43h, and a discharge port 43i. A port 43j in which the upstream branching flow channel 43e and the downstream branching flow channel 43f have opening portions in the same direction is provided in a connection portion with the culture container 35, and the culture container 35 can be attached and detached from a single direction. In addition, the flow channel switching member of the reciprocating motion type is provided inside the storage compartment 43g.

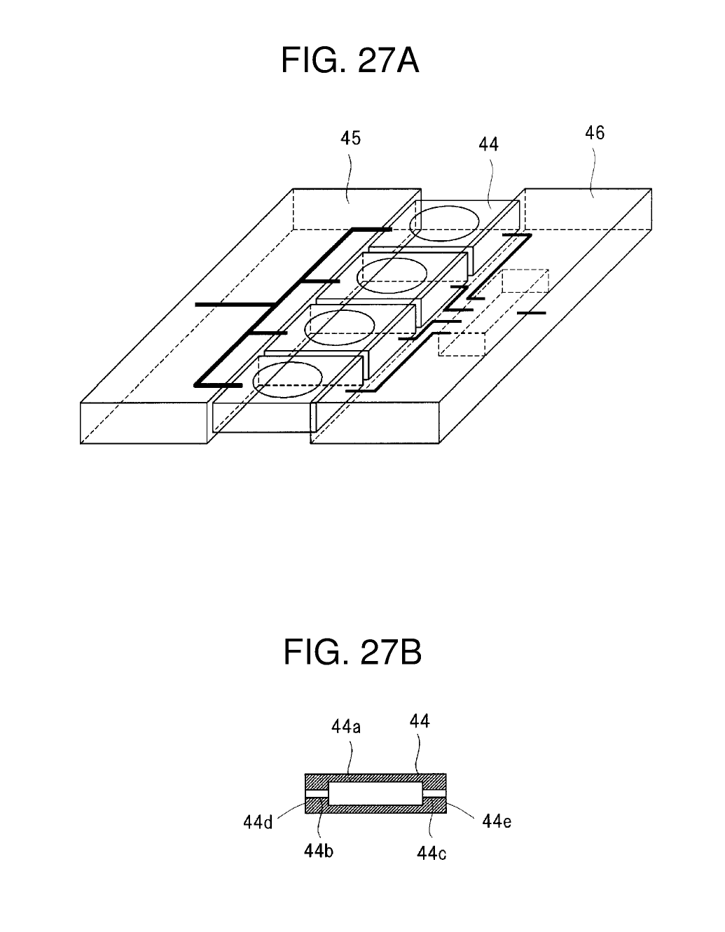

As illustrated in FIG. 27 (a), the integrated flow channel member may include the upstream integrated flow channel member 45 and the downstream integrated flow channel member 46 being divided as separate members. As illustrated in FIG. 27(b), the culture container 44 has a structure which has an inflow channel 44b and a discharge flow channel 44c being drawn out directly opposing each other and two connection ports 44d and 44e with respect to the culture surface 44a, and is connected so as to be interposed between the upstream integrated flow channel member 45 and the downstream integrated flow channel member 46.

The structure as illustrated in FIGS. 27(a) and 27(b) is effective in a case in which it is desired to thin the entire structure although the connection using a culture container having one connection port is difficult and further, the division of member generally leads an increase in cost.

As described above, it is possible to obtain a different effect as follows in addition to the same effects of Embodiment 1 described above according to the cell culture apparatus according to the present embodiment. For example, a representative effect is that it is possible to prevent the occurrence of erroneous piping with respect to the culture container without taking the man-hours in a case in which the plurality of culture containers are connected in parallel using the culture container set which includes the culture container and the integrated flow channel member enclosing the flow channel switching member. Other effects are as described as in the present embodiment.

### Embodiment 4

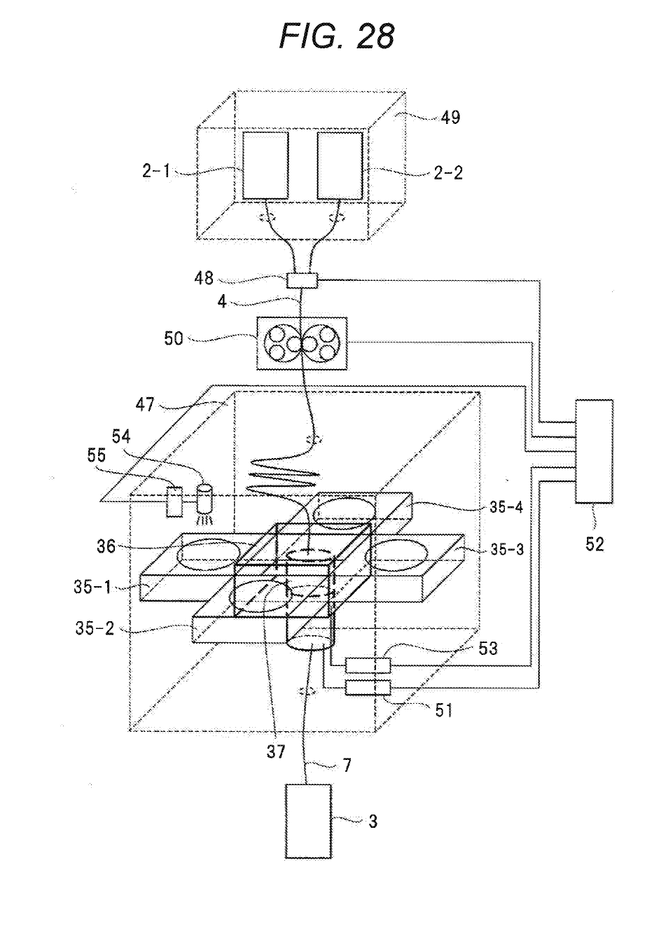

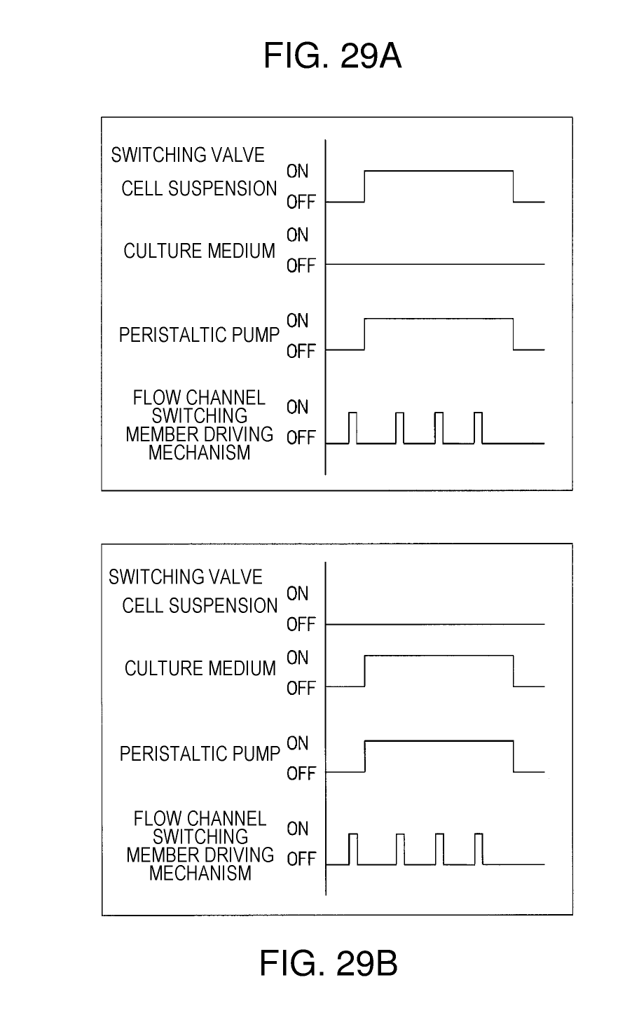

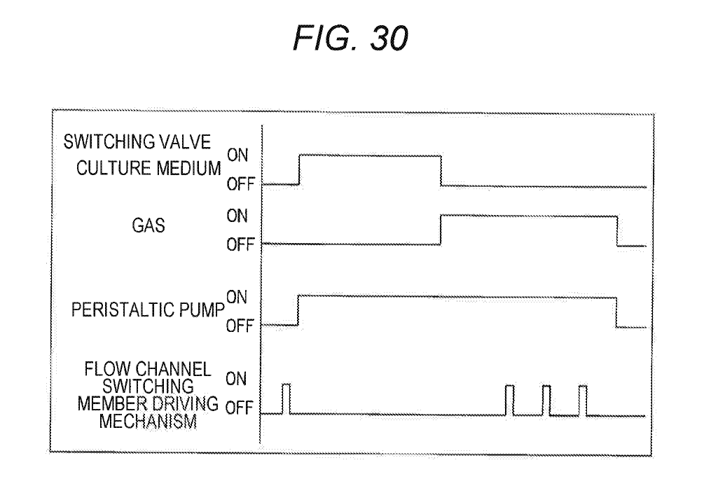

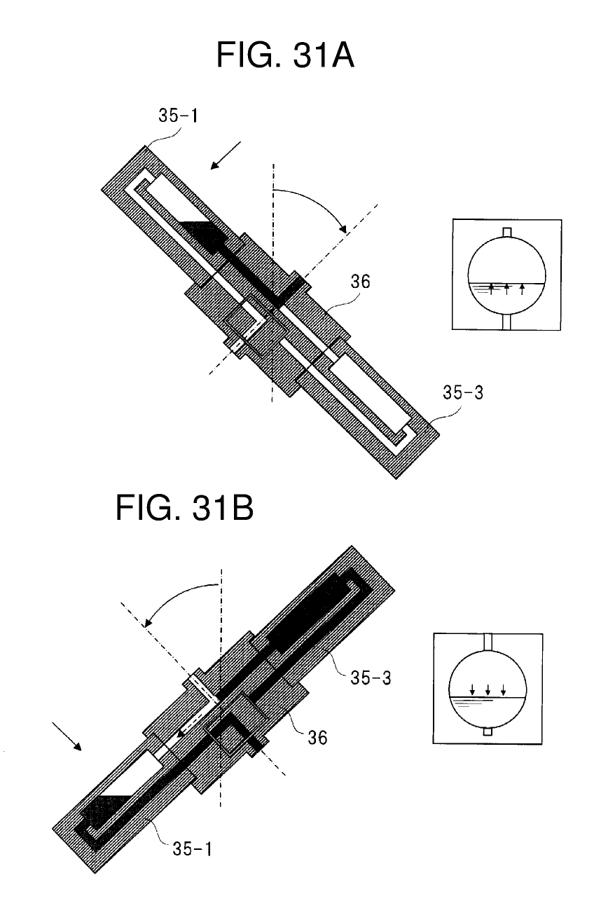

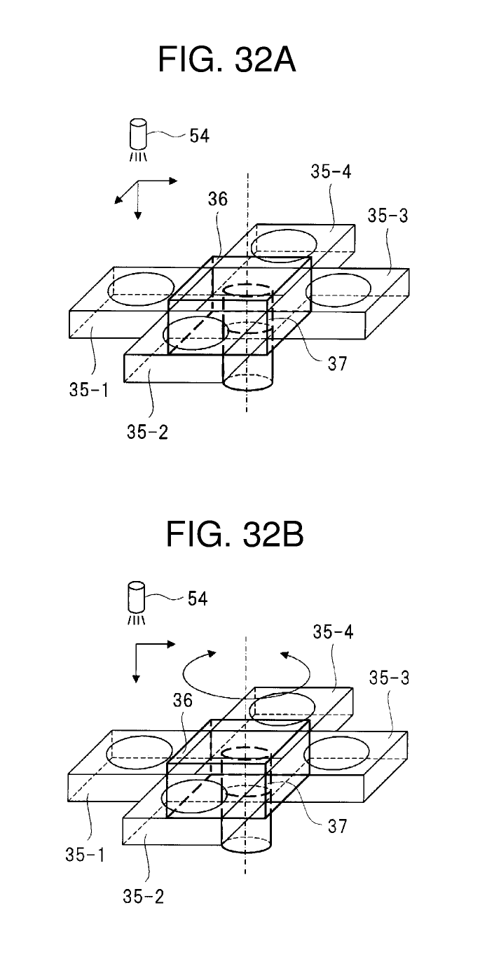

A description will be given regarding a cell culture apparatus according to the present embodiment with reference to FIGS. 28 to 31(b) FIG. 28 is a diagram illustrating an example of an overview of cell culture using a cell culture apparatus. FIGS. 29(a) and 29(b) are diagrams illustrating examples of a control time chart in the cell culture using the cell culture apparatus, and FIG. 30 is a diagram illustrating a modified example thereof. Further, FIGS. 31(a) and 31(b) are FIGS. 31(a) and 31(b) are diagrams illustrating examples of a state of liquid supply in the cell culture using the cell culture apparatus. FIGS. 32(a) and 32(b) are diagrams illustrating examples of drive control of a camera and a culture container in the cell culture using the cell culture apparatus.

A description will be given regarding an example of the cell culture which uses the cell culture apparatus with the closed culture system as described above in the present embodiment. A point that is different from Embodiments 1 to 3 described above will be mainly described also in the present embodiment.

The plurality of culture containers 35-1 to 35-4 are connected to the integrated flow channel member 36 including the flow channel switching member 37 and are installed inside an incubator 47 as an integrated body in FIG. 28. The environment inside the incubator 47 is set in accordance with a type of a culture. For example, the environmental setting in which temperature is 37 degrees, humidity is 95%, and CO₂ concentration is 5% is frequently used.

In addition, common flow channels 4 and 7 are connected to the upstream side and the downstream side of the integrated flow channel member 36, and further, are connected with supply bags 2-1 and 2-2, and the collection bag 3, respectively, thereby forming the closed culture system.

As illustrated in FIG. 28, the supply bags 2-1 and 2-2 may be plural, and are connected to the common flow channel 4. For example, a cell suspension for cell seeding and a culture medium for culture medium replacement may be divided separately, and are prepared in the separate supply bags to be connected with the common flow channel.

These supply bags 2-1 and 2-2 are formed such that the selection of liquid supply can be made by a switching valve 48. This switching means needs to be a switching means different from the means that has been described hereinbefore in order to completely block a flow channel at a location where the flow of liquid is not desired. For example, there is a method of providing a pinch valve for each flow channel, but a description will not be particularly given regarding the method in the present invention. The supply bags 2-1 and 2-2 may be kept in a cool box 49 in order to keep the quality of content.

The collection bag 3 is not particularly limited in terms of the installation location as long as being the drainage, and may be installed also in the cool box in order to delay degradation during a culture period. In addition, a plurality of the collection bags may be connected so as to separate a recovered material using the switching valve similarly as the switching of the liquid supply of the plurality of supply bags although not illustrated.

A peristaltic pump 50, which is a pump mechanism, is installed in the common flow channel 4 as a driving source of liquid supply. The common flow channel 4 is made of silicone rubber, for example, and is capable of supplying the liquid by squeezing the common flow channel 4 using the elasticity thereof.

A flow channel switching member driving mechanism 51, which is a control mechanism, is provided for switching of the flow channel switching member 37, as a mechanism using, for example, a stepping motor or a servomotor which is capable of changing an orientation and a position of the flow channel switching member 37.

A desired liquid supply is realized by controlling the switching valve 48, the peristaltic pump 50, and the flow channel switching member driving mechanism 51 using a control unit 52 in the above-described configuration of the cell culture apparatus.

Next, a description will be given regarding control at the time of seeding a cell, that is, supplying a cell suspension to the culture container. An example of this control time chart is illustrated in FIG. 29(a).

The flow channel switching member 37 is oriented toward the desired culture container (for example, 35-1) using the flow channel switching member driving mechanism 51. The switching valve 48 selects (turns ON) the supply bag (for example, 2-1) containing the cell suspension, and the liquid supply to the desired culture container 35-1 is started when the peristaltic pump 50 is driven (turned ON) in this state. When the liquid is supplied for a predetermined time in this state, and then the culture container 35-1 is filled with the cell suspension, the flow channel switching member 37 is driven and the liquid supply to the different culture container (for example, 35-2) is started. It is possible to perform the seeding to all the plurality of installed culture containers 35-1 to 35-4 by repeating the above-described control. FIGS. 29(a) and 29(b) illustrate examples in which the number of the culture containers is four. Incidentally, the liquid supply may be performed at a certain time, or alternatively, the liquid supply time may be controlled by attaching an arbitrary sensor that detects that the culture container is fully filled with the liquid.

FIG. 29(b) is a control time chart when a culture medium is supplied for the culture medium replacement. The culture medium replacement is executed at regular time intervals during the culture period after seeding the cell. Although the culture medium is selected (turned ON) by the switching valve 48 as a type of the liquid to be supplied, the other operations are almost the same as those at the cell seeding.

Although the content contained in advance is extruded by a liquid to be input from the behind thereof in the present closed culture system, the extruding material is not necessarily the liquid. It may be configured such that a supply bag which contains a sterilized gas is connected instead of the supply bag containing the cell suspension or the culture medium, and the cell suspension or the culture medium contained in advance is pushed out by the gas.

The liquid on the upstream side of the culture container can be used at the subsequent liquid supply, but it is desirable that the amount of such a liquid be minimized as much as possible since the degradation of liquid is accelerated in the state of being placed outside the cool box. When a required amount of liquid is supplied, and the gas is supplied from the behind thereof to extrude the liquid while allowing the liquid to remain inside the cool box, that is, the supply bag as much as possible, the degradation of liquid is little, and it is possible to supply the liquid without waste. FIG. 30 is a control time chart when a required liquid amount of the culture medium is supplied, and then, is extruded by the gas.

Outside air may be taken in via a HEPA filter instead of the supply bag containing the gas. When a diameter of the HEPA filter is set to be small, it can be regarded as a substantially closed system.

Since the liquid supplied from the cool box is cooled, the liquid is preferably supplied to the culture container after being heated to the temperature that does not affect the culture prior to entering the culture container. A heat source dedicated for heating may be provided, but it is more economic to use the heat of environment inside the incubator. It is preferable to form a common flow channel from the entrance of the incubator to the culture container to be long such that the liquid is heated in a stationary state. Volume tanks may be installed instead of the long common flow channel. An arbitrary stirring means may be provided prior to the supply of liquid to the culture container after performing the heating in the stationary state.

Since the culture surface of the culture container is large as compared to the inflow port and the discharge port, air is likely to remain at the time of supplying the liquid from an empty culture container. The liquid may be supplied while tilting the culture container so as to allow the air to pass without remaining in the culture container. FIG. 31(a) illustrates a state in which the liquid is supplied while tilting the culture containers 35-1 and 35-3 (35-2 and 35-4). When the liquid is supplied while tilting the culture container upward, the liquid is supplied to fill the culture container from the bottom due to the gravity, and thus, it is possible to perform the supply without leaving the air. In addition, it is possible to pull out the liquid in the culture container without leaving the liquid in the culture container when the gas is supplied while tilting the culture containers 35-1 and 35-3 (35-2 and 35-4) downward as illustrated in FIG. 31(b).

Since the culture containers 35-1 to 35-4 described in the present embodiment are integrated with the integrated flow channel member 36, it may be enough when the integrated flow channel members 36 is tilted. It is preferable that a tilting mechanism 53 configured for this purpose be simultaneously controlled by the control unit 52 (illustrated in FIG. 28 described above).

In addition, a camera 54 is provided in the incubator 47 as illustrated in FIG. 28 described above, and it is possible to observe a state of the culture inside the culture container. When any difference is confirmed through the result of observation, it is possible to sound an alarm or to change the content of control depending on the content of difference. A camera driving mechanism 55 is attached to the camera 54, and is also controlled by the control unit 52.

Although the plurality of culture containers are provided in the present embodiment, it is desirable to enable the observation of the entire culture container. To do so, the camera 54 needs to be set so as to enable not only a Z-direction control for focusing but also two-dimensional movement in XY directions as illustrated in FIG. 32 (a) There is another method of moving the culture containers 35-1 to 35-4 instead of the camera 54 since it may be enough when facing positions between the camera 54 and each of the culture containers 35-1 to 35-4 are simply changed. An example thereof is illustrated in FIG. 32 (b). As illustrated in FIG. 32 (b), the flow channel switching member driving mechanism may be configured such that the flow channel switching member 37 is fixed and the culture containers 35-1 to 35-4 are moved. In this manner, it is possible to allow an observation system using the camera 54 to be controlled only in a height direction (the Z-control). A uniaxial control (X-control) in a horizontal direction may be added in order for the entire surface observation. Even the added configuration is simpler than a three-axis control, and thus, it is possible to lower the cost.

As described above, it is possible to obtain a different effect as follows in addition to the same effects of Embodiment 1 described above according to the cell culture apparatus according to the present embodiment. For example, a representative effect is that it is possible to select the liquid to be supplied, to maintain the quality of content, and further, to realize a desired liquid supply in the cell culture using the cell culture apparatus. Other effects are as described as in the present embodiment.

Although the description has been given in detail regarding the invention made by the present inventor based on the embodiments as above, the present invention is not limited to the embodiments, and, of course, can be modified in various ways within a scope not departing from a gist thereof. For example, the above-described embodiments have been described in detail in order to describe the present invention in an easily understandable manner, and are not necessarily limited to one including the entire configuration that has been described above. In addition, some configurations of a certain embodiment can be substituted by configurations of another embodiment, and further, a configuration of another embodiment can be added to a configuration of a certain embodiment. In addition, addition, deletion or substitution of other configurations can be made with respect to some configurations of each embodiment.

### [Appended Note]

The present invention has the following characteristics in relation to the culture container set or the like, for example, in addition to the cell culture apparatus described in the claims.

(1) A culture container set to be used in the cell culture apparatus described in the claim (for example, any one of claims 4 to 12), the culture container set including:

a plurality of culture containers having connection ports; and

an integrated flow channel member having a plurality of ports capable of attaching and detaching the plurality of culture containers,

in which the integrated flow channel member includes common flow channels on an upstream side and a downstream side, a plurality of branching flow channels branching from the respective common flow channels and connected to the plurality of ports, and a flow channel switching member, which has a connecting flow channel therein, and is moved to enable communication between a desired flow channel among the plurality of branching flow channels and the connecting flow channel, being enclosed in a branching portion or a merging portion between the common flow channel and the branching flow channel.

(2) A culture container set to be used in the cell culture apparatus described in the claim (for example, claim 13 or 14), the culture container set including:

a plurality of culture containers having connection ports; and

an integrated flow channel member having a plurality of ports capable of attaching and detaching the plurality of culture containers,

in which the integrated flow channel member includes a common flow channel on an upstream side or a downstream side, a plurality of branching flow channels branching from the common flow channel and connected to the plurality of ports, branching flow channels on the downstream side or the upstream side extending from the plurality of ports, and an enclosed flow channel switching member which has a part exposed to the outside of a closed culture system, a sealing portion provided at a boundary thereof, and a connecting flow channel, which serves a common flow channel function, therein, and is moved to enable communication between a desired flow channel among the plurality of branching flow channels the downstream side or the upstream side and the connecting flow channel.

(3) The culture container set described in the above-described (1) or (2), in which each of the culture containers has the single connection port, and is capable of being attached and detached from a single direction.
(4) The culture container set described in any one of the above-described (1) to (3), in which the culture containers have different sizes of culture surfaces while having the same size of the connection ports.
(5) The culture container set described in any one of the above-described (1) to (3), further including a stopper having the same size as the connection port.
(6) The culture container set described in any one of the above-described (1) to (3), in which the culture container, the integrated flow channel member, and the flow channel switching member are resin-molded articles.
(7) A cell culture apparatus described in the claim (for example, any one of claims 4 to 15), the cell culture apparatus including a pump mechanism which controls a supply amount of a liquid depending on a type of a connected culture container.
(8) The cell culture apparatus described in the claim (for example, any one of claims 4 to 15), the cell culture apparatus including a tilting mechanism to tilt a culture container set including the culture container.
(9) The cell culture apparatus described in the claim (for example, any one of claims 4 to 15), the cell culture apparatus including a driving mechanism to fix the flow channel switching member and to drive the culture container side.
(10) The cell culture apparatus described in the claim (for example, any one of claims 4 to 15), the cell culture apparatus including a two-axis control camera driving mechanism which is controlled along an axis in a horizontal direction and an axis in a height direction.
(11) A culture container set including: a plurality of culture containers having connection ports; and

an integrated flow channel member which includes a plurality of ports capable of attaching and detaching the plurality of culture containers, common flow channels on an upstream side and a downstream side, and a plurality of branching flow channels branching from the respective common flow channels and connected to the plurality of ports,

in which each of the culture containers has the single connection port, and is capable of being attached and detached from a single direction.

(12) The culture container set described in the above-described (11), in which the culture containers have different sizes of culture surfaces while having the same size of the connection ports.
(13) The culture container set described in the above-described (11), further including a stopper having the same size as the connection port.
(14) The culture container set described in the above-described (11), in which the culture container, the integrated flow channel member, and the flow channel switching member are resin-molded articles.

### REFERENCE SIGNS LIST

1 . . . culture container
2 . . . supply bag
3 . . . collection bag
4, 4a, 4b, 4p, 4q . . . common flow channel (upstream side)
5, 5a, 5b, 5c . . . branching flow channel (upstream side)
6 . . . branching flow channel (downstream side)
7 . . . common flow channel (downstream side)
8, 8a, 8b . . . flow channel switching mechanism
9 . . . flow channel switching member
9a . . . exposed portion
10 . . . storage compartment
11, 11a, 11b, 11p, 11q . . . connecting flow channel
12 . . . flow channel switching member
13 . . . flow channel switching member
13a . . . inner member
13b . . . rubber
14 . . . groove
15 . . . groove
16, 16p, 16q . . . groove
17 . . . groove
18 . . . O-ring
19 . . . flow channel switching mechanism
20 . . . magnet
21 . . . magnet
22 . . . magnet
23 . . . film
24 . . . concave structure
25 . . . external force transmission mechanism
26 . . . external force transmission mechanism
27 . . . film
28 . . . O-ring
29 . . . flow channel switching member
30 . . . storage compartment
31 . . . through-hole
32 . . . groove
33 . . . groove
34 . . . through-hole
35 . . . culture container
35a . . . culture surface
35b . . . inflow channel
35c . . . discharge flow channel
35d . . . connection port
35e . . . inlet
35f . . . outlet
35p . . . concave structure
35q . . . convex structure
35r . . . taper
36 . . . integrated flow channel member
36a . . . inflow port
36b . . . upstream common flow channel
36c . . . upstream branching flow channel
36d . . . downstream branching flow channel
36e . . . storage compartment
36f . . . downstream common flow channel
36g . . . discharge port
36h . . . port
36p . . . convex structure
36q . . . concave structure
36r . . . taper
36s, 36t . . . dividing member
37 . . . flow channel switching member
38 . . . integrated flow channel member
38a . . . inflow port
38b . . . upstream common flow channel
38c . . . upstream branching flow channel
38d . . . downstream branching flow channel
38e . . . storage compartment
38h . . . port
38p . . . gasket
39 . . . flow channel switching member
39a . . . connecting flow channel
39b . . . discharge port
40 . . . film
41 . . . culture container
42 . . . stopper
43 . . . integrated flow channel member
43a . . . upstream part
43b . . . downstream part
43c . . . inflow port
43d . . . upstream common flow channel
43e . . . upstream branching flow channel
43f . . . downstream branching flow channel
43g . . . storage compartment
43h . . . downstream common flow channel
43i . . . discharge port
43j . . . port
44 . . . culture container
44a . . . culture surface
44b . . . inflow channel
44c . . . discharge flow channel
44d . . . connection port
44e . . . connection port
45 . . . upstream integrated flow channel member
46 . . . downstream integrated flow channel member
47 . . . incubator
48 . . . switching valve
49 . . . cool box
50 . . . peristaltic pump
51 . . . flow channel switching member driving mechanism
52 . . . control unit
53 . . . tilting mechanism
54 . . . camera
55 . . . camera driving mechanism

1. A cell culture apparatus in which a plurality of sealed culture containers having fluid introduction ports and discharge ports are provided, and the plurality of culture containers are connected in parallel to form a single closed culture system, the cell culture apparatus comprising:
a single flow channel switching mechanism which switches a plurality of flow channels connected in the plurality of culture containers.

2. The cell culture apparatus according to claim 1, wherein the flow channel switching mechanism makes a flow channel resistance of one individual flow channel from among the individual flow channels branching and connected to the respective culture containers less than a flow channel resistance of a remainder of the individual flow channels.

3. The cell culture apparatus according to claim 2, wherein the flow channel switching mechanism forms an individual flow channel having a small flow channel resistance by placing a single flow channel switching member in the closed culture system and changing at least one of an orientation and a position of the flow channel switching member.

4. The cell culture apparatus according to claim 3, wherein
the plurality of flow channels branch from, a single upstream common flow channel and are connected to the introduction ports of the respective culture containers, and
the flow channel switching member has at least one connecting flow channel, and is arranged at a branching portion between the upstream common flow channel and the plurality of flow channels,
the cell culture apparatus further comprising a control mechanism which moves the flow channel switching member so as to enable communication between a desired flow channel among the plurality of flow channels and the upstream common flow channel via the connecting flow channel, and to allow the connecting flow channel to communicate with the desired flow channel.

5. The cell culture apparatus according to claim 3, wherein
the plurality of flow channels are merged into a single downstream common flow channel from the discharge ports of the respective culture containers, and
the flow channel switching member has at least one connecting flow channel, and is arranged at a merging portion between the downstream common flow channel and the plurality of flow channels,
the cell culture apparatus further comprising a control mechanism which moves the flow channel switching member so as to enable communication between a desired flow channel among the plurality of flow channels prior to being merged and the downstream common flow channel via the connecting flow channel, and to allow the connecting flow channel to communicate with the desired flow channel.

6. The cell culture apparatus according to claim 4, wherein a space of the branching portion or the merging portion in which the flow channel switching member is arranged is larger than the flow channel switching member.

7. The cell culture apparatus according to claim 4, wherein the flow channel switching member has elasticity in a part or an entire body thereof and is arranged in the branching portion or the merging portion in a state of being contracted.

8. The cell culture apparatus according to claim 4, wherein a structure of the connecting flow channel is a tube structure or a groove structure, or includes the both structures.

9. The cell culture apparatus according to claim 4, wherein the control mechanism rotationally drives the flow channel switching member to cause the connecting flow channel and the desired flow channel to communicate each other.

10. The cell culture apparatus according to claim 4, wherein the control mechanism causes the flow channel switching member to perform reciprocating motion in a single predetermined direction to cause the connecting flow channel and the desired flow channel to communicate each other.

11. The cell culture apparatus according to claim 4, wherein
the flow channel switching member has a fixed first magnetic body, and
the control mechanism has a second magnetic body, and controls the movement of the flow channel switching member using the first magnetic body by moving the second magnetic body.

12. The cell culture apparatus according to claim 4, wherein at least a part of the branching portion or the merging portion in which the flow channel switching member is arranged is provided with a deformable interposed portion such that the movement of the flow channel switching member is controlled by transmitting a force to the flow channel switching member via the interposed portion.

13. The cell culture apparatus according to claim 4, wherein a part of the flow channel switching member is exposed to an outside of the closed culture system, and a sealing member is provided at a boundary between the outside exposed portion and an inside of the closed culture system.

14. The cell culture apparatus according to claim 13, an end of the connecting flow channel inside the flow channel switching member protrudes outward in the outside exposed portion to serve a function also as a common flow channel.

15. The cell culture apparatus according to claim 4, wherein the flow channel switching member has a plurality of connecting flow channels, and performs switching of the plurality of common flow channels.
